(19) United States
(12) Patent Application Publication
(10) Pub. No.: US 2012/0022128 A1
(43) Pub. Date: Jan. 26, 2012

(54) PKIB and NAALADL2 for Target Genes of Prostate Cancer Therapy and Diagnosis

(75) Inventors: Yusuke Nakamura, Tokyo (JP); Hidewaki Nakagawa, Tokyo (JP); Shuichi Nakatsuru, Kanagawa (JP)

(73) Assignee: Oncotherapy Science, Inc., Kanagawa (JP)

(21) Appl. No.: 12/674,664

(22) PCT Filed: Aug. 20, 2008
(86) PCT No.: PCT/JP08/65234

§ 371 Date: Sep. 22, 2010

Related U.S. Application Data
(60) Provisional application No. 60/957,853, filed on Aug. 24, 2007.
(60) Provisional application No. 61/036,030, filed on Mar. 12, 2008.

(51) Int. Cl. A61K 31/713; C12N 15/63; C12Q 1/68; A61P 35/00; C40B 30/04; C07K 16/44; C07K 16/18; G01N 33/566; C12N 15/113; G01N 33/574

(52) U.S. Cl. 514/44 A; 536/24.5; 435/320.1; 435/6.14; 436/501; 435/7.21; 435/7.23; 506/9; 530/387.9; 435/6.13

(57) ABSTRACT

The invention features methods for detecting prostate cancer, especially hormone-refractory prostate cancer (HRPC) or castration-resistant prostate cancer (CRPC), by detecting over-expression of PKIB or NAALADL2 compared the normal organs. Also disclosed are methods of identifying compounds for treating and preventing prostate cancer including HRPC, based on the over-expression of PKIB or NAALADL2 in the prostate cancer, the cell proliferation function of PKIB or NAALADL2, the intracellular localization of PKIB or NAALADL2 or the interaction between PKIB and PKA-C. Also, provided are a method for treating prostate cancer by administering a double-stranded molecule against the PKIB or NAALADL2 gene. The invention also provides products, including the double-stranded molecules and vectors encoding them, as well as compositions comprising the molecules or vectors, useful in the provided methods.

### CROSS REFERENCE TO RELATED APPLICATIONS

This application claims the benefit of U.S. provisional application No. 60/957,853 filed on Aug. 24, 2007, and No. 61/036,030 filed on Mar. 12, 2008. The entire contents of which is hereby incorporated herein by reference for all purposes.

### TECHNICAL FIELD

The present invention relates to the field of biological science, more specifically to the field of cancer diagnosis and treatment. In particular, the present invention relates to methods for detecting and diagnosing prostate cancer as well as methods for treating and preventing prostate cancer. Moreover, the present invention relates to methods for screening an agent for preventing prostate cancer.

### BACKGROUND ART

Prostate cancer (PC) is the most common malignancy in males and the second-leading cause of cancer-related death in the United States and Europe (Gronberg H, Lancet 2003 361:859-64.). The incidence of PC has been increasing significantly in most of developed countries due to prevalence of western-style diet and explosion of the aging population (Gronberg H, Lancet 2003 361:859-64, and Hsing A W et al., Epidemiol Rev 2001 23:3-13.). The screening using serum prostate-specific antigen (PSA) lead to dramatic improvement of early detection of PC and resulted in an increase of the proportion of patients with a localized disease that could be curable by surgical and radiation therapies (Gronberg H, Lancet 2003 361:859-64, and Hsing A W et al., Epidemiol Rev 2001 23:3-13.). However, 20-30% of these PC patients still suffer from the relapse of the disease (Feldman B J et al., Nat Rev Cancer 2001 1:34-45, and Han M et al., J Urol 2001 166:416-9.).

Androgen/androgen receptor (AR) signaling pathway plays the central role in PC development and progression, and the PC growth is usually androgen-dependent at a relatively early stage (Feldman B J et al., Nat Rev Cancer 2001 1:34-45, and Han M et al., J Urol 2001 166:416-9.). Hence, most of the patients with relapsed or advanced disease respond well to androgen-ablation therapy, which suppresses testicular androgen production by surgical or medical castration. Nonetheless, these patients eventually acquire androgen-independent and more aggressive phenotype that has been termed hormone-refractory prostate cancers (HRPCs). Alternatively, they can eventually acquire tolerance to androgen-ablation therapy (castration) and more aggressive phenotypes that are termed castration-resistant prostate cancers (CRPCs), which is basically a lethal disease (Scher H I, Sawyers C L. J Clin Oncol 2006 23:8253-61.). Recently the combination of docetaxel and prednisone was established as the new standard of care for HRPC patients (Tannock I F et al., N Engl J Med 2004 351:1502-12.), but they do not provide a cure and their survival benefit on HRPC patients is very limited. Hence, many groups are now attempting various approaches to identify novel molecule targets or signaling pathways that contribute to growth of HRPC (Scher H I et al., J Clin Oncol 2005 23:8253-61.).

The mechanism of this castration-resistant progression is presumed to be divided into two pathways, those involving AR and those that bypass or are independent of AR. They are not mutually exclusive and frequently co-exist in CRPC cells (Feldman B J, Feldman D. Nat Rev Cancer 2001 1:34-45, Scher H I, Sawyers C L. J Clin Oncol 2006 23:8253-61.). Many AR-bypassing or independent pathways are reported to be activated in CRPC cells, which can contribute to their more malignant or aggressive phenotype. Cross-talk between the AR pathway and independent pathways, such as Her-2/neu and IL-6/STATS, can also occur (Feldman B J, Feldman D. Nat Rev Cancer 2001 1:34-45, Scher H I, Sawyers C L. J Clin Oncol 2006 23:8253-61, Grossmann M E, et al. JNCI-2001 93:1687-97, Yang L, et al. BBRC 2003 305: 462-469.).

Among the independent pathways, PTEN-PI3K-Akt pathway is likely to be one of the most critical pathways that can explain CRPC phenotype. Akt is a serine/threonine kinase which is activated by phosphatidylinositol (3,4,5)-phosphate (PIP3), and activated or phosphorylated Akt promotes both cell growth and cell survival by regulating GSK3beta, BAD, FOXO and mTOR (Sharma M, et al. J Biol Chem 2002 277: 30935-41, Pap M, Cooper G M. J Biol Chem 1998 273:19929-32, Downward J. 1998 Curr Opin Cell Biol 10:262-67, Datta S R, et al. Cell 1997 91: 231-41, Downward J. Cell Develop Biol 2004 15:177-82, Vivanco I and Sawyers C. Nat Rev Cancer 2002 2: 289-501, Hay N. Cancer Cell 2005 8:179-83).

In normal cells, the tumor suppressor PTEN, a lipid phosphatase that removes phosphate from PIP3, inhibits Akt activation and allows the cells to undergo apoptosis, while some tumor cells harbor a PTEN mutation or loss of PTEN expression, leading to Akt activation. In addition to its anti-apoptotic function, in prostate cancer cells, activated Akt directly binds to AR and phosphorylates AR in the absence of androgen, which can also contribute to CRPC phenotypes (Wen Y, et al. Cancer Res 2000 60: 6841-45).

In fact, the level of phosphorylated Akt was elevated in high Gleason grade PC and it was associated with PC progression or CRPC progression (Malik S N, et al. Clin Cancer Res 2002 8: 1168-71, Kreisberg J I, et al. Cancer Res 2004 64:5232-36).

Akt requires phosphorylation at Thr308 and Ser473 residues for its activation. Phosphoinositide-dependent kinase 1 (PDK1) can catalyze the phosphorylation of Thr308, and a number of kinases have been suggested to function as the so-called PDK2 which catalyze the phosphorylation of Ser473, but whether any or all of them act as a physiological PDK2 in cancer cells remains to be established (Grossmann M E, et al. JNCI-2001 93:1687-97, Tasken K, Aandahl E M. Physol Review 2004 84: 137-67.).

On the other hand, cAMP-dependent protein kinase A (PKA) is often considered essential for mediating the wide range of physiological or pathological effects initiated by cAMP, and coupling with G-proteins. A number of ligand-receptor systems activate PKA signaling pathway, and its activation is associated with the control of cell growth and differentiation (Tasken K, Aandahl E M. Physol Review 2004 84: 137-67, Stork P J, Schmitt J M. Trends Cell Biol 2002 12:258-66).

In prostate cancer, several reports suggested the involvement of PKA with androgen independent growth and neuroendocrine differentiation (Cox M E, et al. J Biol Chem 2000 275: 13812-8, Deeble P D, Cox M E, et al. Cancer Res 2007 67:663-72). Cross-talk between the PKA pathway and the AR pathway is also suggested to be involved with androgen-independent or castration-resistant growth of PC cells (Stork P J, Schmitt J M. Trends Cell Biol 2002 12:258-66, Sadar M D. J Biol Chem 1999 274: 7777-83).

This PKA pathway is regulated by many kinds of factors like PKA-regulatory subunits (PKA-R) or PKA inhibitors (Taylor S S, Kim C, Vigil D, et al. BBA 2005 1754: 25-37, Dalton G D, Dewey W L. Neuropeptides 2006 40: 23-34), and in cancer cells, these regulatory factors are aberrantly expressed to modify PKA pathway and some of them are targeted for cancer treatment (Miller W R. Ann NY Acad Sci 2002 968: 37-48, 2002).

Previously, in order to characterize the molecular features of clinical HRPCs or CRPCs and identify the molecular targets for HRPC or CRPC treatment, genome-wide cDNA microarray analysis was performed for cancer cells purified from HRPC or CRPC tissues by means of LMM (laser microbeam microdissection) and a number of de-regulated genes were identified in HRPC or CRPC cells, some of which might be involved in androgen-independence and aggressive phenotypes (Tamura K et al., Cancer Res 2007 67: 5117-25.).

### SUMMARY OF THE INVENTION

Based on genome-wide expression profiles of HRPC or CRPC cells, two molecular targets, PKIB (GenBank accession number: NM_{—}181795) and NAALADL2 (GenBank accession number: NM_{—}207015 and AK021754) have been identified as useful for PC treatment and diagnosis. In addition, the proteins are useful as molecular targets for development of novel treatments for HRPC.

PKIB is one of the regulatory factors in the PKA pathway, as an over-expressing gene in CRPC. The inventors have demonstrated that it contributes to PC cell viability and its malignant phenotype through functional linking between the PKA pathway and the Akt pathway.

PKIB belongs to PKI (protein kinase A inhibitor) family. PKIA is thought to inhibit the kinase activity of protein kinase A catalytic subunit (PKA-C) (GenBank accession number: NM_{—}002730) and export PKA-C from the nucleus to the cytoplasm by binding to PKA-C directly (Glass D B et al., J Biol Chem 1986 261: 12166-71, and Wen W et al., J Biol Chem 1994 269: 32214-20.). Protein kinase A (PKA), cAMP-dependent protein kinase A, is often considered essential for mediating the wide range of physiological or pathological effects initiated by cAMP, and coupling with G protein, a number of ligand and receptor systems activate PKA signaling pathway, and its activation is associated with the control of cell growth and differentiation (Tasken K et al., Physol Review 2004 84: 137-67, and Stork P J et al., Trends Cell Biol 2002 12:258-66.). In prostate cancer, several reports suggested its involvement with androgen-independent growth and neuroendocrine differentiation (Cox M E et al., J Biol Chem 2000 275: 13812-8.), and cross-talk between PKA pathway and AR pathway is suggested to be involved with androgen-independent growth of HRPC cells (Stork P T et al., Trends Cell Biol 2002 12:258-66, and Sadar M D, J Biol Chem 1999 274: 7777-83.).

NAALADL2 is a novel type II membrane protein and belongs to glutamate carboxypeptidase II (GCPII) family. The prostate form of GCPII, termed prostate-specific membrane antigen (PSMA) is expressed in prostate cancer and increased levels of PSMA are associated with PC progression and HRPC (Rajasekaran A K et al., Am J Physiol Cell Physiol 2005 288: C975-81, and Murphy G P et al., Prostate 2000 42: 145-9.). Considering its homology with PSMA and its similar expression pattern, NAALADL2 should be termed as “PSMA2”. PSMA is the target of an FDA-approved prostate cancer-imaging agent, the ¹¹¹In-labeled 7E11 monoclonal antibody (Prostascint, Cytogen, Princeton, N.J.). PSMA is targeted by monoclonal antibodies such as J591, which is in clinical trials for specific delivery of imaging agent or therapeutics to PSMA-expressing cells (Murphy G P et al., Prostate 2000 42: 145-9, and Holmes E H, Expert Opin Investig Drugs 2001 10: 511-9.). In addition to its character as a tumor marker, PSMA has GPC activity whose substrates include poly-γ-glutamated folates (Zhou J et al., Nature Review Drug Disc 2005 4: 1015-26.). The enzymatic activity of PSMA can be exploited for the design of prodrugs, in which an inactive glutamated form of the drug is selectively cleaved and thereby activated only at cells expressing PSMA (Denny W A et al., Eur J Med Chem 2001 36: 577-95.). However, how PSMA is associated with prostate cancer progression is completely unknown, and possibility of targeting PSMA function or activity itself is yet to be known.

The present invention features a method of diagnosing or determining a predisposition to prostate cancer in a subject by determining an expression level of PKIB and NAALADL2 in a patient derived biological sample. An increase of the expression level of any of the genes compared to a normal control level of the genes indicates that the subject suffers from or is at risk of developing prostate cancer.

The present invention is based, at least in part, on the discovery that double-stranded molecules comprising specific sequences (in particular, SEQ ID NOs: 16, 17 and 19) are effective for inhibiting cellular growth of prostate cancer cells. Specifically, small interfering RNAs (siRNAs) targeting PKIB and NAALADL2 genes are provided by the present invention.

According to an aspect of the present invention, the double-stranded molecules may be encoded in vectors and expressed from the vectors.

Thus, the invention provides methods for inhibiting cell growth and treating prostate cancer by administering the double-stranded molecules or vectors of the present invention. Such methods include administering to a subject a composition comprising one or more of the double-stranded molecules or vectors.

Another aspect of the invention relates to compositions for treating cancer containing at least one of the double-stranded molecules or vectors of the present invention. Alternatively, the invention further provides a method of screening for a compound for treating or preventing prostate cancer, which includes the steps of contacting a test compound with a cell that expresses PKIB or NAALADL2 protein and then selecting the test compound that reduces the expression level of the PKIB or NAALADL2 protein. Furthermore, the present invention provides a method of screening for a compound for treating or preventing prostate cancer wherein the binding between PKIB and protein kinase A catalytic subunit (PKA-C) is detected. Compounds that inhibit the binding between PKIB and PKA-C are expected to reduce a symptom of prostate cancer. Moreover, the present invention provides a method of screening for an antibody for detecting cancer wherein NAALADL2 is detected in the cell surface. The antibody that recognized the NAALADL2 is used for detecting prostate cancer.

### BRIEF DESCRIPTION OF THE DRAWINGS

FIG. 1A Semi-quantitative RT-PCR confirmed PKIB overexpression in HRPC cells (5/5), but not in HSPC cells, compared with normal prostatic epithelial cells which were also microdissected (NPmix), whole normal prostate tissue, and vital organs (heart, lung, liver, and kidney). ACTB was used to quantify the each of cDNA contents. B Multiple Tissue Northern (MTN) blot analysis for PKIB expression showed about 1.5-kb band in placenta, but not in vital organs (heart, lung, liver, and kidney), among the human adult organs. Northern blot analysis for PKIB expression showed that several PC cell lines (22Rv1 and PC-3) strongly expressed PKIB, while other normal adult organs did not express PKIB. C-F depict the immunohistochemical analysis on PC tissues. C prostatic intraepithelial neoplasia (PIN) showed week staining (+) for PKIB. D PC with Gleason Grade 3 showed week staining (+), while normal prostate epithelium (N) showed negative staining. E PC with Gleason Grade 5 showed strong positive staining (+++) for PKIB. F HRPC also showed strong positive staining (+++) for PKIB.

FIG. 2 A Semi-quantitative RT-PCR confirmed NAALADL2 overexpression in HRPC cells (7/11), compared with normal prostatic epithelial cells which were also microdissected (NPmix), whole normal prostate tissue, and vital organs (heart, lung, liver, and kidney). ACTB was used to quantify the each of cDNA contents. B MTN blot analysis for NAALADL2 expression showed three bands of about 10-kb, 6-kb, and 5-kb only in PC cell lines, but not in vital organs (heart, lung, liver, and kidney), among the human adult organs.

FIG. 3 Effect of PKIB-siRNA on growth of PC cells. A RT-PCR confirmed knockdown effect on PKIB expression by si1 and si2, but not by si3 and a negative control siEGFP in 22Rv1 (left) and LNCaP(HP) cells (right). ACTB was used to quantify RNAs. B MTT assay of each of 22Rv1 (left) and LNCaP(HP) cells (right) transfected with indicated siRNA-expressing vectors to PKIB (si1, si2 and si3) and a negative control vector (siEGFP). Each average is plotted with error bars indicating SD (standard deviation) after 20 days incubation with Geneticin. ABS on Y-axis means absorbance at 490 nm, and at 630 nm as reference, measured with a microplate reader. These experiments were carried out in triplicate. Transfected with si1 and si2 in of 22Rv1 (left) and LNCaP(HP) cells (right) resulted in a drastic reduction in the number of viable cells, compared with si3 and siEGFP for which no knockdown effect was observed (P<0.01, Student's t-test). C Colony formation assay of 22Rv1 (left) and LNCaP(HP) cells (right) transfected with each of indicated siRNA-expressing vectors to PKIB (si1, si2 and si3) and a negative control vector (siEGFP). Cells were visualized with 0.1% crystal violet staining after 20 days incubation with Geneticin.

FIG. 4 Effect of NAALADL2-siRNA on growth of PC cells. A RT-PCR confirmed knockdown effect on NAALADL2 expression by si#690, but not by si#913, si#1328 and a negative control siEGFP in 22Rv1 cells. ACTB was used to quantify RNAs. B MTT assay of each of 22Rv1 cells transfected with indicated siRNA-expressing vectors to NAALADL2 (si#690, si#913, and si#1328) and a negative control vector (siEGFP). Each average is plotted with error bars indicating SD (standard deviation) after 20 days incubation with Geneticin. Y-axis means absorbance at 490 nm, and at 630 nm as reference, measured with a microplate reader. These experiments were carried out in triplicate. Transfected with si#690 in 22Rv1 cells resulted in a drastic reduction in the number of viable cells, compared with other siRNA for which no knockdown effect was observed (P<0.01, Student's t-test). C Colony formation assay of 22Rv1 cells transfected with each of indicated siRNA-expressing vectors to NAALADL2 (si#690, si#913, and si#1328) and a negative control vector (siEGFP). Cells were visualized with 0.1% crystal violet staining after 20 days incubation with Geneticin. D RT-PCR confirmed knockdown effect on NAALADL2 expression by synthesized RNA duplex corresponding to si#690 in another NAALADL2-expressing C4-2B cell. ACTB was used to quantify RNAs. E The synthesized RNA duplex corresponding to si#690 suppressed the cell viability of C4-2B cells, compared with a control RNA duplex siEGFP (P<0.01, Student's t-test).

FIG. 5 Subcellular localization of PKIB (A) NAALADL2 (B) proteins. Immuocytochemical analysis using anti-tag antibody showed that exogenous PKIB was localized at the cytoplasm and exogenous NAALADL2 protein was mainly localized at the cytoplasmic membrane. C PKIB-Myc and HA-PKA-C expression vectors were co-transfected to 22Rv1 cells, and their cell lysates were immunoprecipitated by each of tag antibody. PKIB-Myc was co-immunoprecipitaed with PKA-C and vice versa, indicating the direct interaction between PKIB and PKA-C. D Immunocytochemical analysis observed most of PKA-C was localized in the cytoplasm and some signal of PKA-C protein in the nucleus (left) when control siRNA transfected to PC-3 cells. On the other hand, when siRNA knocked down endogenous PKIB in PC-3 cells, immunocytochemical analysis showed no or very little signal of PKA-C in the nucleus (right). E After siRNA duplex was treated in PC cells, the cells were fractionated to the nuclear and the cytoplasmic fractions to analyze the nuclear PKA-C more quantitatively. 30 micro grams protein of the fractionated cell lysates was western-blotted by using anti-PKA-C antibody and anti-laminB antibody for the loading and nucleus-fractionated control. The amount of PKA-C in the nucleus was clearly decreased in PKIB knockdown by siRNA, comparing with that in control siRNA, while the amount of PKA-C in the cytoplasm was a little increased in PKIB knockdown.

FIG. 6 A RT-PCR validated the constitutive expression of PKIB in DU145-derived clones (PKIB #1, #2, and #3). B Western blot analysis validated the constitutive expression of PKIB in DU145-derived clones (PKIB #1, #2, and #3). C In-vitro growth rate of DU145 clones expressing high level of exogenous PKIB (clones 1-3) and those transfected with mock vector (the mixture of #1, #2, #3 mixture). X-, and Y-axis represent day point after seeding and relative growth rate that was calculated in absorbance of the diameter by comparison with the absorbance value of day 1 as a control. PKIB-overexpressing cells grew more rapidly than mock cells, suggesting the growth-promoting effect of PKIB in prostate cancer. D 2×10⁶ DU145 cells stably expressed PKIB (right) or Mock cells (left) were inoculated to the flanks of male nude mice. 15 weeks after inoculation, tumors were established only on the right side (PKIB++; arrows), but not on the left side (Mock). E Matrigel invasion assay demonstrating the invasive nature of NIH3T3 cells after transfection with PKIB expression vector. Y-axis represents the number of cells migrating through the Matrigel-coated filters. Assays were carried out three times, and each average is plotted with error bars indicating SD (standard deviation). Over-expression of PKIB significantly promoted the invasive nature of NIH3T3 cells (P=0.0052).

FIG. 7A Knockdown of PKIB by siRNA duplex (siPKIB) in LNCaP and PC-3 cells resulted in attenuated phosphorylation at Ser 473 of Akt. Transfection of siEGFP duplex was served as a negative control. Knockdown of PKIB was validated by RT-PCR and ACTB was served as a loading control. B Overexpression of PKIB enhanced the phosphorylation at Ser 473 of Akt in PC-3 and 22Rv1 cells. PKIB overexpression was confirmed by western blot using HA-tag antibody. C Overexpression of PKA-C also enhanced the phosphorylation at Ser 473 of Akt in PC-3 cells. PKA-C overexpression was confirmed by western blot using HA-tag antibody and total amount of Akt was served as a loading control. D In vitro kinase assay of Akt using recombinant PKIB and PKA-C proteins. The phosphorylation of Akt-Ser473 was detected by anti-phospho-Akt (Ser473) antibody (Cell Signaling), and total amount of Akt was detected by anti-Akt antibody. PKIB addition to PKA-C kinase significantly increased the phosphorylation of Akt-Ser473 in vitro.

FIG. 8 PKIB expression was correlated with Akt phosphorylation in clinical PC tissues. The pictures represent immunohistochemistry on the face-to-face slides of PC tissues for A PKIB and for B phosphorylated Akt.

### THE DISCLOSURE OF THE INVENTION

### Definition

The words “a”, “an”, and “the” as used herein mean “at least one” unless otherwise specifically indicated.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or polynucleotides.

The term “polynucleotide” and “oligonucleotide” are used interchangeably herein unless otherwise specifically indicated and are referred to by their commonly accepted single-letter codes. The terms apply to nucleic acid (nucleotide) polymers in which one or more nucleic acids are linked by ester bonding. The polynucleotide or oligonulceotide may be composed of DNA, RNA or a combination thereof.

### Double-Stranded Molecule

The term “isolated double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As use herein, the term “siRNA” refers to a double-stranded RNA molecule which prevents translation of a target mRNA. Standard techniques of introducing siRNA into the cell are used, including those in which DNA is a template from which RNA is transcribed. The siRNA includes a PKIB or NAALADL2 sense nucleic acid sequence (also referred to as “sense strand”), a PKIB or NAALADL2 antisense nucleic acid sequence (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules comprising complementary sequences to one another and that have annealed together via the complementary sequences to form a double-stranded RNA molecule. The nucleotide sequence of two strands may comprise not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, comprising a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotied composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a PKIB or NAALADL2 sense nucleic acid sequence (also referred to as “sense strand”), a PKIB or NAALADL2 antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules comprising complementary sequences to one another and that have annealed together via the complementary sequences to form a double-stranded polynucleotide molecule. The nucleotide sequence of two strands may comprise not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, comprising a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, an “isolated nucleic acid” is a nucleic acid removed from its original environment (e.g., the natural environment if naturally occurring) and thus, synthetically altered from its natural state. In the present invention, isolated nucleic acid includes DNA, RNA, and derivatives thereof.

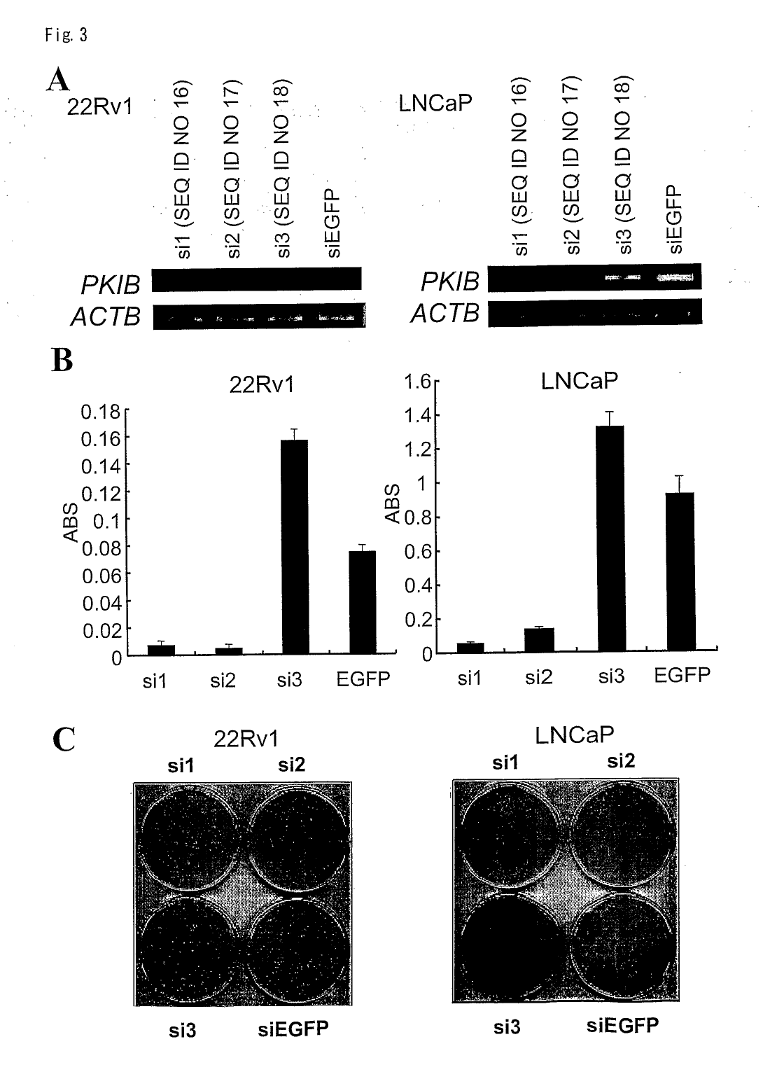

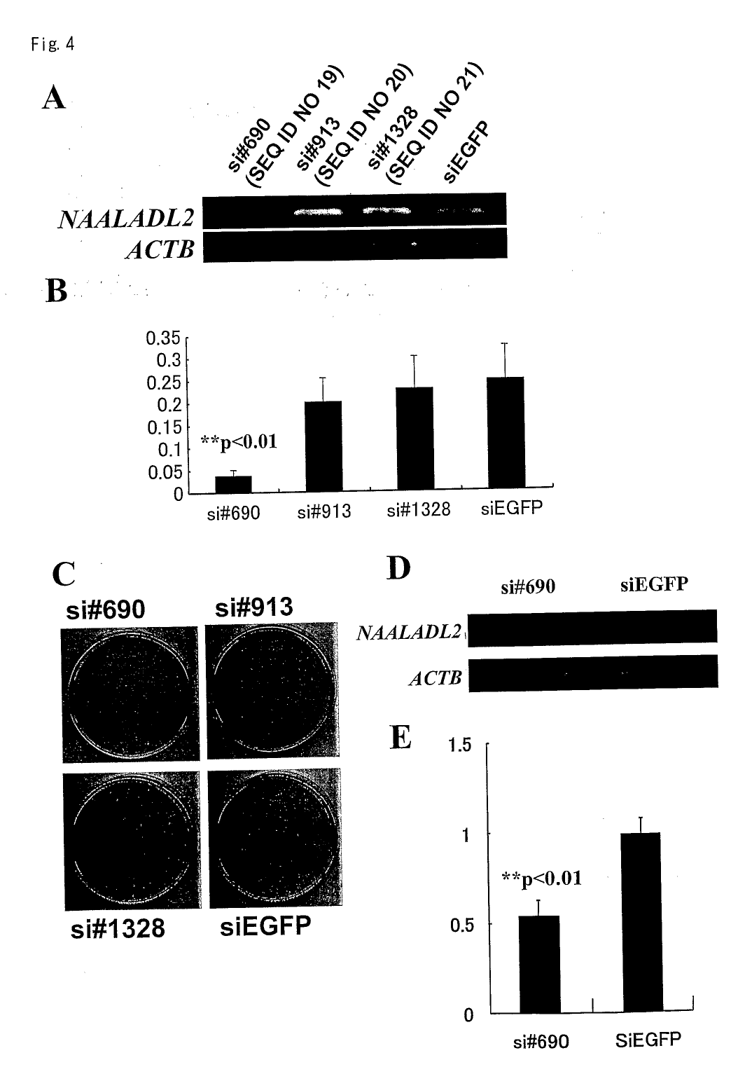

A double-stranded molecule against PKIB or NAALADL2, which molecule hybridizes to target mRNA, decreases or inhibits production of PKIB or NAALADL2 protein encoded by PKIB or NAALADL2 gene by associating with the normally single-stranded mRNA transcript of the gene, thereby interfering with translation and thus, inhibiting expression of the protein. The expression of PKIB in prostate cancer cell lines, was inhibited by 1 or 2 different dsRNA (FIG. 3); the expression of NAALADL2 in prostate cancer cell lines was inhibited by dsRNA (FIG. 4).

Therefore the present invention provides isolated double-stranded molecules having the property to inhibit expression of PKIB or NAALADL2 gene when introduced into a cell expressing the gene. The target sequence of double-stranded molecule is designed by siRNA design algorithm mentioned below.

PKIB target sequence includes, for example, nucleotides
SEQ ID NO: 16,
SEQ ID NO: 17

NAALADL2 target sequence includes, for example, nucleotides
SEQ ID NO: 19

Specifically, the present invention provides the following double-stranded molecules [1] to [16]:
[1] An isolated double-stranded molecule, when introduced into a cell, inhibits expression of a PKIB or NAALADL2 gene and cell growth, which molecule comprises a sense strand and an antisense strand complementary thereto, hybridized to each other to form the double-stranded molecule and, wherein the sense strand comprises a target sequence selected from the group consisting of SEQ ID NOs: 16, 17 and 19;
[2] The double-stranded molecule of [1], which has a length of less than about 100 nucleotides;
[3] The double-stranded molecule of [2], which has a length of less than about 75 nucleotides;
[4] The double-stranded molecule of [3], which has a length of less than about 50 nucleotides;
[5] The double-stranded molecule of [4] which has a length of less than about 25 nucleotides;
[6] The double-stranded molecule of [5], which has a length of between about 19 and about 25 nucleotides;
[7] The double-stranded molecule of [1], which consists of a single polynucleotide comprising both the sense and antisense strands linked by an intervening single-strand;
[8] The double-stranded molecule of [7], which has the general formula 5′-[A]-[B]-[A′]-3′, wherein [A] is the sense strand comprising a sequence selected from the group consisting of SEQ ID NOs: 16, 17 and 19, [B] is the intervening single-strand consisting of 3 to 23 nucleotides, and [A′] is the antisense strand comprising a complementary sequence to [A];
[9] The double-stranded molecule of [1], which comprises RNA;
[10] The double-stranded molecule of [1], which comprises both DNA and RNA;
[11] The double-stranded molecule of [10], which is a hybrid of a DNA polynucleotide and an RNA polynucleotide;
[12] The double-stranded molecule of [11] wherein the sense and the antisense strands consist of DNA and RNA, respectively;
[13] The double-stranded molecule of [10], which is a chimera of DNA and RNA;
[14] The double-stranded molecule of [13], wherein a region flanking to the 3′-end of the antisense strand, or both of a region flanking to the 5′-end of sense strand and a region flanking to the 3′-end of antisense strand consists of RNA;
[15] The double-stranded molecule of [14], wherein the flanking region consists of 9 to 13 nucleotides; and
[16] The double-stranded molecule of [1], which contains 3′ overhang.

The double-stranded molecule of the present invention will be described in more detail below.

Methods for designing double-stranded molecules having the ability to inhibit target gene expression in cells are known. (See, for example, U.S. Pat. No. 6,506,559, herein incorporated by reference in its entirety). For example, a computer program for designing siRNAs is available from the Ambion website (http://www.ambion.com/techlib/misc/siRNA_finder.html).

The computer program selects target nucleotide sequences for double-stranded molecules based on the following protocol.

### Selection of Target Sites

1. Beginning with the AUG start codon of the transcript, scan downstream for AA di-nucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.
2. Compare the potential target sites to the appropriate genome database (human, mouse, rat, etc.) and eliminate from consideration any target sequences with significant homology to other coding sequences. Basically, BLAST, which can be found on the NCBI server at: www.ncbi.nlm.nih.gov/BLAST/, is used (Altschul S F et al., Nucleic Acids Res 1997 Sep. 1, 25(17): 3389-402).
3. Select qualifying target sequences for synthesis. Selecting several target sequences along the length of the gene to evaluate is typical.

By the protocol, the target sequence of the isolated double-stranded molecules of the present invention were designed as
SEQ ID NO: 16 and
SEQ ID NO: 17 for PKIB gene; and
SEQ ID NO: 19 for NAALADL2 gene.

Double-stranded molecules targeting the above-mentioned target sequences were respectively examined for their ability to suppress the growth of cells expressing the target genes. Therefore, the present invention provides double-stranded molecules targeting any of the sequences selected from the group of
SEQ ID NO: 16 and
SEQ ID NO: 17 for PKIB gene; and
SEQ ID NO: 19 for NAALADL2 gene.

The double-stranded molecule of the present invention is directed to a single target PKIB or NAALADL2 gene sequence or may be directed to a plurality of target PKIB or NAALADL2 gene sequences.

By PKIB or NAALADL2 target sequence is meant a nucleotide sequence that is identical to a portion of the PKIB gene or the NAALADL2 gene (i.e, a polynucleotide within a PKIB or NAALADL2 gene that is equal in length to and complementary to an siRNA). The target sequence can include the 5′ untranslated (UT) region, the open reading frame (ORF) or the 3′ untranslated region of the human PKIB or NAALADL2 gene. Alternatively, the siRNA is a nucleic acid sequence complementary to an upstream or downstream modulator of PKIB or NAALADL2 gene expression. Examples of upstream and downstream modulators include, a transcription factor that binds the PKIB or NAALADL2 gene promoter, a kinase or phosphatase that interacts with the PKIB or NAALADL2 polypeptide, a PKIB or NAALADL2 promoter or enhancer.

A double-stranded molecule of the present invention targeting the above-mentioned targeting sequence of PKIB or NAALADL2 gene include isolated polynucleotides that comprises any of the nucleic acid sequences of target sequences and/or complementary sequences to the target sequences. Examples of polynucleotides targeting PKIB gene include those comprising the sequence of SEQ ID NO: 16 or 17 and/or complementary sequences to these nucleotides; polynucleotides targeting NAALADL2 gene include those comprising the sequence of SEQ ID NO: 19 and/or complementary sequences to these nucleotides. However, the present invention is not limited to these examples, and minor modifications in the aforementioned nucleic acid sequences are acceptable so long as the modified molecule retains the ability to suppress the expression of PKIB or NAALADL2 gene. Herein, “minor modification” in a nucleic acid sequence indicates one, two or several substitutions, deletions, additions or insertions of nucleic acids to the sequence. Typically, a minor modification will be four or fewer, sometimes three or fewer, and often two or fewer substitutions, deletions, additions or insertions of nucleic acids to the sequence.

According to the present invention, a double-stranded molecule of the present invention can be tested for its ability using the methods utilized in the Examples. In the Examples, the double-stranded molecules comprising sense strands or antisense strands complementary thereto of various portions of mRNA of PKIB or NAALADL2 genes were tested in vitro for their ability to decrease production of PKIB or NAALADL2 gene product in prostate cancer cell lines (e.g., using 22Rv1, LNCaP(HP) and C4-2B) according to standard methods. Furthermore, for example, reduction in PKIB or NAALADL2 gene product in cells contacted with the candidate double-stranded molecule compared to cells cultured in the absence of the candidate molecule can be detected by, e.g. RT-PCR using primers for PKIB or NAALADL2 mRNA mentioned under Example 1, item “Semi-quantitative RT-PCR”. Sequences which decrease the production of PKIB or NAALADL2 gene product in in vitro cell-based assays can then be tested for their inhibitory effects on cell growth. Sequences which inhibit cell growth in in vitro cell-based assay can then be tested for their in vivo ability using animals with cancer, e.g. nude mouse xenograft models, to confirm decreased production of PKIB or NAALADL2 product and decreased cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide comprises modified nucleotides and/or non-phosphodiester linkages, these polynucleotides may also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The polynucleotide is less than 1909 nucleotides in length for PKIB and less than 4912 nucleotides in length for NAALADL2. For example, the polynucleotide is less than 500, 200, 100, 75, 50, or 25 nucleotides in length for all of the genes. The isolated polynucleotides of the present invention are useful for forming double-stranded molecules against PKIB or NAALADL2 gene or preparing template DNAs encoding the double-stranded molecules. When the polynucleotides are used for forming double-stranded molecules, the polynucleotide may be longer than 19 nucleotides, preferably longer than 21 nucleotides, and more preferably has a length of between about 19 and 25 nucleotides.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include phosphorothioate linkages, 2′-β-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxyabasic residue incorporation (US20060122137). In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule consisting of a DNA strand (polynucloeotide) and an RNA strand (polynucleotide), a chimera type double-stranded molecule comprising both DNA and RNA on any or both of the single strands (polynucleotides), or the like may be formed for enhancing stability of the double-stranded molecule. The hybrid of a DNA strand and an RNA strand may be either where the sense strand is DNA and the antisense strand is RNA, or the opposite so long as it has an activity to inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule preferably contains as much DNA as possible, whereas to induce inhibition of the target gene expression, the molecule is required to be RNA within a range to induce sufficient inhibition of the expression. As a preferred example of the chimera type double-syanded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in preferable embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded molecule of the present invention comprise following combinations.
sense strand: 5′-[DNA]-3′
   3′-(RNA)-[DNA]-5′: antisense strand,
sense strand: 5′-(RNA)-[DNA]-3′
   3′-(RNA)-[DNA]-5′: antisense strand, and
sense strand: 5′-(RNA)-[DNA]-3′
   3′-(RNA)-5′: antisense strand.

The upstream partial region preferably is a domain consisting of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA comprises the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence consisting of an arbitrary nucleotide sequence can be located between the sense and antisense sequence in order to form the hairpin loop structure. Thus, the present invention also provides a double-stranded molecule having the general formula 5′-[A]-[B]-[A′]-3′, wherein [A] is the sense strand comprising a target sequence, [B] is an intervening single-strand and [A′] is the antisense strand comprising a complementary sequence to [A]. The target sequence may be selected from the group consisting of, for example, nucleotides
SEQ ID NO: 16, or
SEQ ID NO: 17 for PKIB; and
SEQ ID NO: 19 for NAALADL2.

The present invention is not limited to these examples, and the target sequence in [A] may be modified sequences from these examples so long as the double-stranded molecule retains the ability to suppress the expression of the targeted PKIB or NAALADL2 gene. The region [A] hybridizes to [A′] to form a loop consisting of the region [B]. The intervening single-stranded portion [B], i.e., loop sequence may be preferably 3 to 23 nucleotides in length. The loop sequence, for example, can be selected from group consisting of following sequences (http://www.ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn D M et al., Nat Rev Mol Cell Biol 2003 June, 4(6): 457-67.

Exemplary, preferable double-stranded molecules having hairpin loop structure of the present invention are shown below. In the following structure, the loop sequence can be selected from group consisting of AUG, CCC, UUCG, CCACC, CTCGAG, AAGCUU, CCACACC, and UUCAAGAGA; however, the present invention is not limited thereto:

(for target sequence SEQ ID NO: 16)
gauaugccaucccagauuu-[B]-aaaucugggauggcauauc;
(for target sequence SEQ ID NO: 17)
gucaaauuccccaaauuaa-[B]-uuaauuuggggaauuugac;
and
(for target sequence SEQ ID NO: 19)
guguccagaggccaauauu-[B]-aauauuggccucuggacac;

Furthermore, in order to enhance the inhibition activity of the double-stranded molecules, nucleotide “u” can be added to 3′ end of the antisense strand of the target sequence, as 3′ overhangs. The number of “u”s to be added is at least 2, generally 2 to 10, preferably 2 to 5. The added “u”s form single strand at the 3′ end of the antisense strand of the double-stranded molecule.

The method of preparing the double-stranded molecule is not particularly limited but it is preferable to use a chemical synthetic method known in the art. According to the chemical synthesis method, sense and antisense single-stranded polynucleotides are separately synthesized and then annealed together via an appropriate method to obtain a double-stranded molecule. Specific example for the annealing includes wherein the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking PKIB or NAALADL2 sequences may be identical or different, such that their expression can be modulated independently, or in a temporal or spatial manner. The double-stranded molecules can be transcribed intracellularly by cloning PKIB or NAALADL2 gene templates into a vector containing, e.g., a RNA pol III transcription unit from the small nuclear RNA (snRNA) U6 or the human H1 RNA promoter.

### Vector Containing the Double-Stranded Molecule

Also included in the invention is a vector containing one or more of the double-stranded molecules described herein, and a cell containing the vector. A vector of the present invention preferably encodes a double-stranded molecule of the present invention in an expressible form. Herein, the phrase “in an expressible form” indicates that the vector, when introduced into a cell, will express the molecule. In a preferred embodiment, the vector includes regulatory elements necessary for expression of the double-stranded molecule. Such vectors of the present invention may be used for producing the present double-stranded molecules, or directly as an active ingredient for treating cancer.

Vectors of the present invention can be produced, for example, by cloning PKIB or NAALADL2 sequence into an expression vector so that regulatory sequences are operatively-linked to PKIB or NAALADL2 sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the double-stranded molecule are utilized to respectively express the sense and anti-sense strands and then forming a double-stranded molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

The vectors of the present invention may be, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

### Methods of Treating Cancer Using the Double-Stranded Molecule

In present invention, 3 different dsRNA for PKIB and 3 different dsRNA for NAALADL2 were constructed to test for their ability to inhibit cell growth. The two dsRNA for PKIB effectively knocked down the expression of the gene in two prostate cancer cell lines coincided with suppression of cell proliferation (FIGS. 3A, B and C). The one dsRNA for NAALADL2 significantly decreased the expression level and cell growth activity in prostate cell line (FIG. 4A to E).

Therefore, the present invention provides methods for inhibiting cell growth, i.e., prostate cancer cell growth, by inducing dysfunction of PKIB or NAALADL2 gene via inhibiting the expression of PKIB or NAALADL2 gene. PKIB or NAALADL2 gene expression can be inhibited by any of the aforementioned double-stranded molecules of the present invention which specifically target of PKIB or NAALADL2 gene or the vectors of the present invention that can express any of the double-stranded molecules.

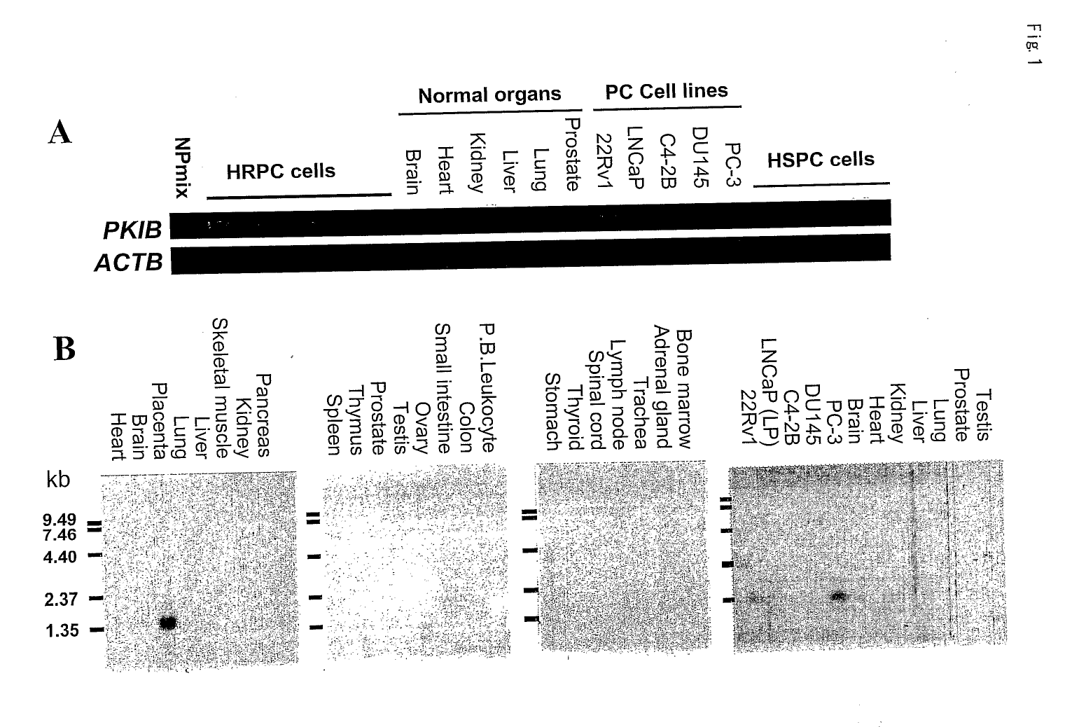

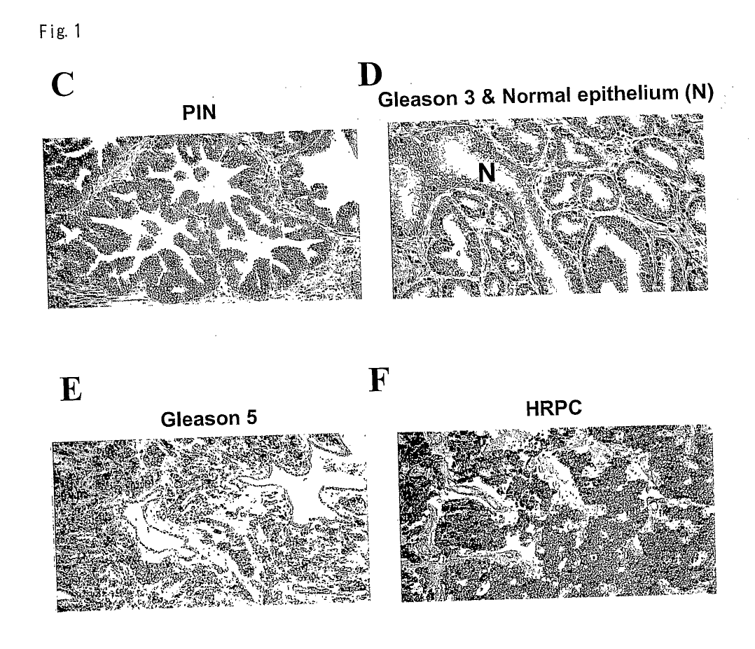

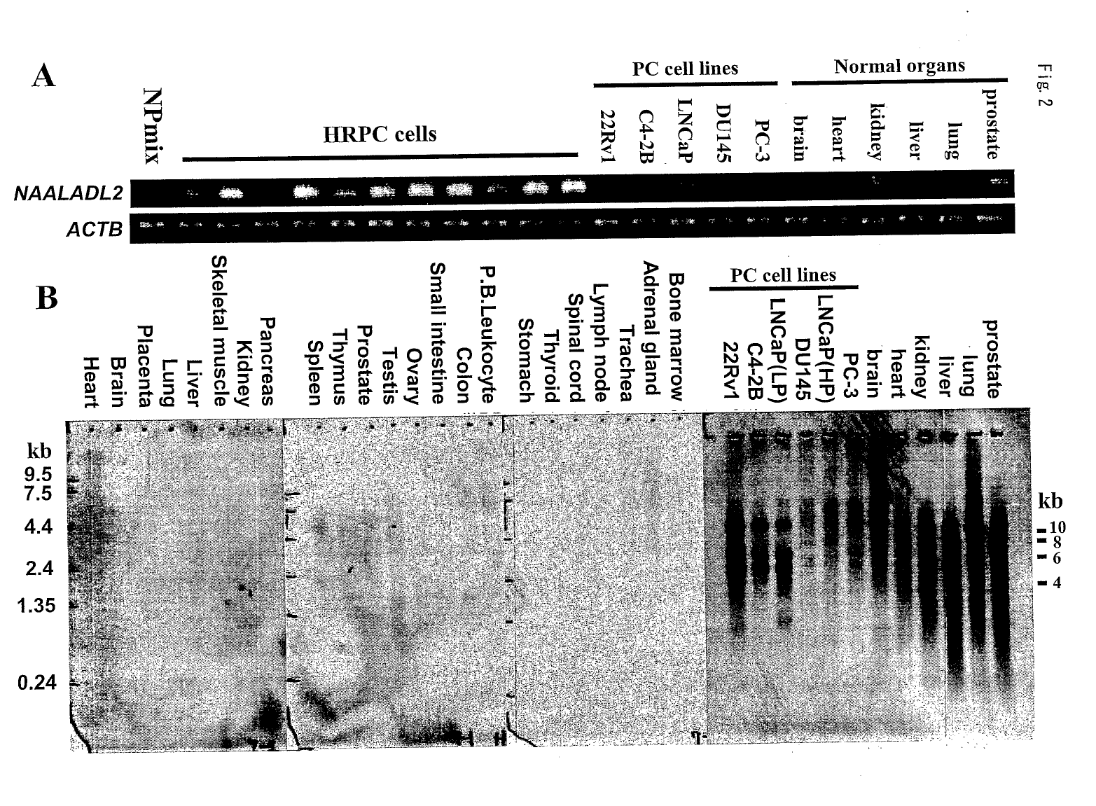

Such ability of the present double-stranded molecules and vectors to inhibit cell growth of cancerous cell indicates that they can be used for methods for treating cancer. Thus, the present invention provides methods to treat patients with prostate cancer by administerning a double-stranded molecule against PKIB or NAALADL2 gene or a vector expressing the molecule without adverse effect because that genes were hardly detected in normal organs (FIGS. 1 and 2).

The term “specifically inhibit” in the context of inhibitory polynucleotides and polypeptides refers to the ability of an agent or ligand to inhibit the expression or the biological function of PKIB or NAALADL2. Specific inhibition typically results in at least about a 2-fold inhibition over background, preferably greater than about 10-fold and most preferably greater than 100-fold inhibition of PKIB or NAALADL2 expression (e.g., transcription or translation) or measured biological function (e.g., cell growth or proliferation, inhibition of apoptosis). Expression levels and/or biological function can be measured in the context of comparing treated and untreated cells, or a cell population before and after treatment. In some embodiments, the expression or biological function of PKIB or NAALADL2 is completely inhibited. Typically, specific inhibition is a statistically meaningful reduction in PKIB or NAALADL2 expression or biological function (e.g., p 0.05) using an appropriate statistical test.

Specifically, the present invention provides the following methods [1] to [23]:
[1] A method for treating prostate cancer comprising the step of administering at least one isolated double-stranded molecule inhibiting the expression of PKIB or NAALADL2 in a cell over-expressing the gene and the cell proliferation, which molecule comprises a sense strand and an antisense strand complementary thereto, hybridized to each other to form the double-stranded molecule.
[2] The method of [1], wherein the sense strand comprises the sequence corresponding to a target sequence selected from the group consisting of SEQ ID NOs: 16, 17 and 19.
[3] The method of [1], wherein the prostate cancer to be treated is hormone-refractory prostate cancer or castration-resistant prostate cancer;
[4] The method of [1], wherein plural kinds of the double-stranded molecules are administered;
[5] The method of [4], wherein the plural kinds of the double-stranded molecules target the same gene;
[6] The method of [1], wherein the double-stranded molecule has a length of less than about 100 nucleotides;
[7] The method of [6], wherein the double-stranded molecule has a length of less than about 75 nucleotides;
[8] The method of [7], wherein the double-stranded molecule has a length of less than about 50 nucleotides;
[9] The method of [8], wherein the double-stranded molecule has a length of less than about 25 nucleotides;
[10] The method of [9], wherein the double-stranded molecule has a length of between about 19 and about 25 nucleotides in length;
[11] The method of [1], wherein the double-stranded molecule consists of a single polynucleotide comprising both the sense strand and the antisense strand linked by an interventing single-strand;
[12] The method of [11], wherein the double-stranded molecule has the general formula 5′-[A]-[B]-[A′]-3′, wherein [A] is the sense strand comprising a sequence selected from the group consisting of SEQ ID NOs: 16, 17 and 19, [B] is the intervening single strand consisting of 3 to 23 nucleotides, and [A′] is the antisense strand comprising a complementary sequence to [A];
[13] The method of [1], wherein the double-stranded molecule comprises RNA;
[14] The method of [1], wherein the double-stranded molecule comprises both DNA and RNA;
[15] The method of [14], wherein the double-stranded molecule is a hybrid of a DNA polynucleotide and an RNA polynucleotide;
[16] The method of [15] wherein the sense and antisense strand polynucleotides consist of DNA and RNA, respectively;
[17] The method of [14], wherein the double-stranded molecule is a chimera of DNA and RNA;
[18] The method of [17], wherein a region flanking to the 3′-end of the antisense strand, or both of a region flanking to the 5′-end of sense strand and a region flanking to the 3′-end of antisense strand consists of RNA;
[19] The method of [18], wherein the flanking region consists of 9 to 13 nucleotides;
[20] The method of [1], wherein the double-stranded molecule contains 3′ overhangs;
[21] The method of [1], wherein the double-stranded molecule is encoded by a vector;
[22] The method of [21], wherein the double-stranded molecule encoded by the vector has the general formula 5′-[A]-[BHA']-3′, wherein [A] is the sense strand comprising a sequence selected from the group consisting of SEQ ID NOs: 16, 17 and 19, [B] is a intervening single-strand consisting of 3 to 23 nucleotides, and [A′] is the antisense strand comprising a complementary sequence to [A]; and
[23] The method of [1], wherein the double-stranded molecule is contained in a composition which comprises in addition to the molecule a transfection-enhancing agent and pharmaceutically acceptable carrier.

The method of the present invention will be described in more detail below.

The growth of cells expressing PKIB or NAALADL2 gene is inhibited by contacting the cells with a double-stranded molecule against PKIB or NAALADL2 gene, a vector expressing the molecule or a composition comprising the same. The cell is further contacted with a transfection agent. Suitable transfection agents are known in the art. The phrase “inhibition of cell growth” indicates that the cell proliferates at a lower rate or has decreased viability compared to a cell not exposed to the molecule. Cell growth may be measured by methods known in the art, e.g., using the MTT cell proliferation assay.

The growth of any kind of cell may be suppressed according to the present method so long as the cell expresses or over-expresses the target gene of the double-stranded molecule of the present invention. Exemplary cells include prostate cancer cells.

Thus, patients suffering from or at risk of developing disease related to PKIB or NAALADL2 may be treated by administering at least one of the present double-stranded molecules, at least one vector expressing at least one of the molecules or at least one composition comprising at least one of the molecules. For example, patients of prostate cancer may be treated according to the present methods. The type of cancer may be identified by standard methods according to the particular type of tumor to be diagnosed. Prostate cancer may be diagnosed, for example, with prostate-specific antigen (PSA) or digital rectal exam. More preferably, patients treated by the methods of the present invention are selected by detecting the expression of PKIB or NAALADL2 in a biopsy from the patient by RT-PCR or immunoassay. Preferably, before the treatment of the present invention, the biopsy specimen from the subject is confirmed for PKIB or NAALADL2 gene over-expression by methods known in the art, for example, immunohistochemical analysis or RT-PCR.

According to the present method to inhibit cell growth and thereby treat cancer, when administering plural kinds of the double-stranded molecules (or vectors expressing or compositions containing the same), each of the molecules may be directed to the same target sequence, or different target sequences of PKIB and/or NAALADL2. For example, the method may utilize double-stranded molecules directed to PKIB or NAALADL2. Alternatively, for example, the method may utilize double-stranded molecules directed to one, two or more target sequences selected from PKIB and NAALADL2.

For inhibiting cell growth, a double-stranded molecule of present invention may be directly introduced into the cells in a form to achieve binding of the molecule with corresponding mRNA transcripts. Alternatively, as described above, a DNA encoding the double-stranded molecule may be introduced into cells as a vector. For introducing the double-stranded molecules and vectors into the cells, transfection-enhancing agent, such as FuGENE (Rochediagnostices), Lipofectamine 2000 (Invitrogen), Oligofectamine (Invitrogen), and Nucleofector (Wako pure Chemical), may be employed.

A treatment is determined efficacious if it leads to clinical benefit such as, reduction in expression of PKIB or NAALADL2 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

Prevention and prophylaxis include any activity which reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis include a wide range of prophylactic therapies aimed at alleviating the severity of the particular disorder, e.g. reducing the proliferation and metastasis of tumors, reducing angiogenesis.

Treating and/or for the prophylaxis of cancer or, and/or the prevention of postoperative recurrence thereof includes any of the following steps, such as surgical removal of cancer cells, inhibition of the growth of cancerous cells, involution or regression of a tumor, induction of remission and suppression of occurrence of cancer, tumor regression, and reduction or inhibition of metastasis. Effectively treating and/or the prophylaxis of cancer decreases mortality and improves the prognosis of individuals having cancer, decreases the levels of tumor markers in the blood, and alleviates detectable symptoms accompanying cancer.

It is understood that the double-stranded molecule of the invention degrades the target mRNA (PKIB or NAALADL2) in substoichiometric amounts. Without wishing to be bound by any theory, it is believed that the double-stranded molecule of the invention causes degradation of the target mRNA in a catalytic manner. Thus, compared to standard cancer therapies, significantly less double-stranded molecule needs to be delivered at or near the site of cancer to exert therapeutic effect.

One skilled in the art can readily determine an effective amount of the double-stranded molecule of the invention to be administered to a given subject, by taking into account factors such as body weight, age, sex, type of disease, symptoms and other conditions of the subject; the route of administration; and whether the administration is regional or systemic. Generally, an effective amount of the double-stranded molecule of the invention comprises an intercellular concentration at or near the cancer site of from about 1 nanomolar (nM) to about 100 nM, preferably from about 2 nM to about 50 nM, more preferably from about 2.5 nM to about 10 nM. It is contemplated that greater or smaller amounts of the double-stranded molecule can be administered.

The present methods can be used to inhibit the growth or metastasis of cancer; for example prostate cancer, especially hormone-refractory prostate cancer or castration-resistant prostate cancer. In particular, a double-stranded molecule comprising a target sequence of PKIB (i.e., SEQ ID NOs: 16 and 17) is particularly preferred for the treatment of prostate cancer; those comprising a target sequence of NAALADL2 (i.e., SEQ ID NOs: 19) is particularly preferred for the treatment of prostate cancer.

For treating cancer, the double-stranded molecule of the invention can also be administered to a subject in combination with a pharmaceutical agent different from the double-stranded molecule. Alternatively, the double-stranded molecule of the invention can be administered to a subject in combination with another therapeutic method designed to treat cancer. For example, the double-stranded molecule of the invention can be administered in combination with therapeutic methods currently employed for treating cancer or preventing cancer metastasis (e.g., radiation therapy, surgery and treatment using chemotherapeutic agents, such as cisplatin, carboplatin, cyclophosphamide, 5-fluorouracil, adriamycin, daunorubicin or tamoxifen).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with the present a double-stranded molecule include the Mirus Transit TKO® lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as prostate tumor tissue, and can also increase the blood half-life of the double-stranded molecule. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded molecule comprises a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

Particularly preferably, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH. sub. 3 and a solvent mixture such as tetrahydrofuran and water in a 30:12 ratio at 60 degrees C.

Vectors expressing a double-stranded molecule of the invention are discussed above. Such vectors expressing at least one double-stranded molecule of the invention can also be administered directly or in conjunction with a suitable delivery reagent, including the Minis Transit LT1® lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecule of the invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection); subcutaneous injection or deposition including subcutaneous infusion (such as by osmotic pumps); direct application to the area at or near the site of cancer, for example by a catheter or other placement device (e.g., a suppository or an implant comprising a porous, non-porous, or gelatinous material); and inhalation. It is preferred that injections or infusions of the double-stranded molecule or vector be given at or near the site of cancer.

The double-stranded molecule of the invention can be administered in a single dose or in multiple doses. Where the administration of the double-stranded molecule of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded molecule of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, more preferably from about seven to about ten days. In a preferred dosage regimen, the double-stranded molecule is injected at or near the site of cancer once a day for seven days. Where a dosage regimen comprises multiple administrations, it is understood that the effective amount of a double-stranded molecule administered to the subject can comprise the total amount of a double-stranded molecule administered over the entire dosage regimen.

### Compositions Comprising the Double-Stranded Molecule

Furthermore, the present invention provides pharmaceutical compositions comprising at least one of the present double-stranded molecules or the vectors coding for the molecules. Specifically, the present invention provides the following compositions [1] to [23]:
[1] A composition for treating prostate cancer, comprising at least one isolated double-stranded molecule inhibiting the expression of PKIB or NAALADL2 and the cell proliferation, which molecule comprises a sense strand and an antisense strand complementary thereto, hybridized to each other to form the double-stranded molecule.
[2] The composition of [1], wherein the sense strand comprises the sequence corresponding to a target sequence selected from the group consisting of SEQ ID NOs: 16, 17 and 19.
[3] The composition of [1], wherein the prostate cancer to be treated is hormone-refractory prostate cancer or castration-resistant prostate cancer;
[4] The composition of [1], wherein the composition contains plural kinds of the double-stranded molecules;
[5] The composition of [4], wherein the plural kinds of the double-stranded molecules target the same gene;
[6] The composition of [1], wherein the double-stranded molecule has a length of less than about 100 nucleotides;
[7] The composition of [6], wherein the double-stranded molecule has a length of less than about 75 nucleotides;
[8] The composition of [7], wherein the double-stranded molecule has a length of less than about 50 nucleotides;
[9] The composition of [8], wherein the double-stranded molecule has a length of less than about 25 nucleotides;
[10] The composition of [9], wherein the double-stranded molecule has a length of between about 19 and about 25 nucleotides;
[11] The composition of [1], wherein the double-stranded molecule consists of a single polynucleotide comprising the sense strand and the antisense strand linked by an intervening single-strand;
[12] The composition of [11], wherein the double-stranded molecule has the general formula 5′-[A]-[B]-[A′]-3′, wherein [A] is the sense strand sequence comprising a sequence selected from the group consisting of SEQ ID NOs: 16, 17 and 19, [B] is the intervening single-strand consisting of 3 to 23 nucleotides, and [A′] is the antisense strand comprising a complementary sequence to [A];
[13] The composition of [1], wherein the double-stranded molecule comprises RNA;
[14] The composition of [1], wherein the double-stranded molecule comprises DNA and RNA;
[15] The composition of [14], wherein the double-stranded molecule is a hybrid of a DNA polynucleotide and an RNA polynucleotide;
[16] The composition of [15], wherein the sense and antisense strand polynucleotides consist of DNA and RNA, respectively;
[17] The composition of [14], wherein the double-stranded molecule is a chimera of DNA and RNA;
[18] The composition of [17], wherein a region flanking to the 3′-end of the antisense strand, or both of a region flanking to the 5′-end of sense strand and a region flanking to the 3′-end of antisense strand consists of RNA;
[19] The composition of [18], wherein the flanking region consists of 9 to 13 nucleotides;
[20] The composition of [1], wherein the double-stranded molecule contains 3′ overhangs;
[21] The composition of [1], wherein the double-stranded molecule is encoded by a vector and contained in the composition;
[22] The composition of [21], wherein the double-stranded molecule has the general formula 5′-[A]-[B]-[A′]-3′, wherein [A] is the sense strand comprising a sequence selected from the group consisting of SEQ ID NOs: 16, 17 and 19, [B] is a intervening single-strand consisting of 3 to 23 nucleotides, and [A′] is the antisense strand comprising a complementary sequence to [A]; and
[23] The composition of [1], wherein the composition comprises a transfection-enhancing agent and pharmaceutically acceptable carrier.

The method of the present invention will be described in more detail below.

The double-stranded molecules of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise at least one of the double-stranded molecules or vectors encoding them of the present invention (e.g., 0.1 to 90% by weight), or a physiologically acceptable salt of the molecule, mixed with a physiologically acceptable carrier medium. Preferred physiologically acceptable carrier media are water, buffered water, normal saline, 0.4% saline, 0.3% glycine, hyaluronic acid and the like.

According to the present invention, the composition may contain plural kinds of the double-stranded molecules, each of the molecules may be directed to the same target sequence, or different target sequences of PKIB and/or NAALADL2. For example, the composition may contain double-stranded molecules directed to PKIB or NAALADL2. Alternatively, for example, the composition may contain double-stranded molecules directed to one, two or more target sequences selected from PKIB and NAALADL2.

Furthermore, the present composition may contain a vector coding for one or plural double-stranded molecules. For example, the vector may encode one, two or several kinds of the present double-stranded molecules. Alternatively, the present composition may contain plural kinds of vectors, each of the vectors coding for a different double-stranded molecule.

Moreover, the present double-stranded molecules may be contained as liposomes in the present composition. See under the item of “Methods of treating cancer using the double-stranded molecule” for details of liposomes.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25-75%, of one or more double-stranded molecule of the invention. A pharmaceutical composition for aerosol (inhalational) administration can comprise 0.01-20% by weight, preferably 1-10% by weight, of one or more double-stranded molecule of the invention encapsulated in a liposome as described above, and propellant. A carrier can also be included as desired; e.g., lecithin for intranasal delivery.

In addition to the above, the present composition may contain other pharmaceutical active ingredients so long as they do not inhibit the in vivo function of the present double-stranded molecules. For example, the composition may contain chemotherapeutic agents conventionally used for treating cancers.

In another embodiment, the present invention also provides the use of the double-stranded nucleic acid molecules of the present invention in manufacturing a pharmaceutical composition for treating a cancer expressing the PKIB and/or NAALADL2 gene(s). For example, the present invention relates to a use of double-stranded nucleic acid molecule inhibiting the expression of a PKIB and/or NAALADL2 gene(s) in a cell, which molecule comprises a sense strand and an antisense strand complementary thereto, hybridized to each other to form the double-stranded nucleic acid molecule and targets to a sequence selected from the group consisting of SEQ ID NOs: 16, 17 and 19, for manufacturing a pharmaceutical composition for treating a cancer expressing the PKIB and/or NAALADL2 gene(s).

Alternatively, the present invention further provides a method or process for manufacturing a pharmaceutical composition for treating a cancer expressing the PKIB and/or NAALADL2 gene(s), wherein the method or process comprises step for formulating a pharmaceutically or physiologically acceptable carrier with a double-stranded nucleic acid molecule inhibiting the expression of a PKIB and/or NAALADL2 gene(s) in a cell, which molecule comprises a sense strand and an antisense strand complementary thereto, hybridized to each other to form the double-stranded nucleic acid molecule and targets to a sequence selected from the group consisting of SEQ ID NOs: 16, 17 and 19 as active ingredients.

In another embodiment, the present invention also provides a method or process for manufacturing a pharmaceutical composition for treating a cancer expressing the PKIB and/or NAALADL2 gene(s), wherein the method or process comprises step for admixing an active ingredient with a pharmaceutically or physiologically acceptable carrier, wherein the active ingredient is a double-stranded nucleic acid molecule inhibiting the expression of a PKIB and/or NAALADL2 gene(s) in a cell, which molecule comprises a sense strand and an antisense strand complementary thereto, hybridized to each other to form the double-stranded nucleic acid molecule and targets to a sequence selected from the group consisting of SEQ ID NOs: 16, 17 and 19.

Alternatively, according to the present invention, use of the double-stranded molecule of this invention for manufacturing a pharmaceutical composition for treating prostate cancer is provided. Further, the present invention also provides the double-stranded molecule of the present invention for treating prostate cancer.

### A Method for Diagnosing Prostate Cancer

The expression of PKIB or NAALADL2 was found to be specifically elevated in prostate cancer cells (FIG. 1A, B, C, D, E, F and FIG. 2A, B). Therefore, the gene identified herein as well as its transcription and translation products find diagnostic utility as a marker for prostate cancer and by measuring the expression of PKIB or NAALADL2 in a cell sample, prostate cancer can be diagnosed. Specifically, the present invention provides a method for diagnosing prostate cancer by determining the expression level of PKIB or NAALADL2 in the subject. The prostate cancers that can be diagnosed by the present method include hormone-refractory prostate cancers or castration-resistant prostate cancers.

The methods of the present invention may provide an initial result for determining the condition of a subject. Such initial results may be combined with additional information to assist a doctor, nurse, or other practitioner to diagnose the disease. Alternatively, the present invention may be used to detect cancerous cells in a subject-derived tissue, and provide a doctor with useful information to diagnose the disease.

Specifically, the present invention provides the following methods [1] to [10]:
[1] A method for diagnosing or detecting the presence of prostate cancer, said method comprising the steps of:
   (a) detecting the expression level of the gene encoding the amino acid sequence of PKIB or NAALADL2 in a biological sample; and
   (b) relating an increasing in the expression level as compared to a normal control level of the gene to the disease.
[2] The method of [1], wherein the expression level is at least 10% greater than the normal control level.
[3] The method of [1], wherein the expression level is detected by any one of the method selected from the group consisting of:
   (a) detecting the mRNA comprising the sequence of PKIB or NAALADL2,
   (b) detecting the protein comprising the amino acid sequence of PKIB or NAALADL2, and
   (c) detecting the biological activity of the protein comprising the amino acid sequence of PKIB or NAALADL2.
[4] The method of [1], wherein the prostate cancer is hormone-refractory prostate cancer or castration-resistant prostate cancer.
[5] The method of [3], wherein the expression level is determined by detecting hybridization of a probe to a gene transcript of the gene.
[6] The method of [3], wherein the expression level is determined by detecting the binding of an antibody against the protein encoded by a gene as the expression level of the gene.
[7] The method of [1], wherein the biological sample comprises biopsy, sputum blood or urine.
[8] The method of [1], wherein the subject-derived biological sample comprises an epithelial cell.
[9] The method of [1], wherein the subject-derived biological sample comprises a cancer cell.
[10] The method of [1], wherein the subject-derived biological sample comprises a cancerous epithelial cell.

The method of diagnosing or detecting the presence of prostate cancer will be described in more detail below.

A subject to be diagnosed by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the determination so long as it comprises the objective transcription or translation product of PKIB or NAALADL2. The biological samples include, but are not limited to, bodily tissues and fluids, such as blood, sputum and urine. Preferably, the biological sample contains a cell population comprising an epithelial cell, more preferably a cancerous epithelial cell or an epithelial cell derived from prostate tissue suspected to be cancerous. Further, if necessary, the cell may be purified from the obtained bodily tissues and fluids, and then used as the biological sample.

According to the present invention, the expression level of PKIB or NAALADL2 in the subject-derived biological sample is determined. The expression level can be determined at the transcription (nucleic acid) product level, using methods known in the art. For example, the mRNA of PKIB or NAALADL2 may be quantified using probes by hybridization methods (e.g., Northern hybridization). The detection may be carried out on a chip or an array. The use of an array is preferable for detecting the expression level of a plurality of genes (e.g., various cancer specific genes) including PKIB or NAALADL2. Those skilled in the art can prepare such probes utilizing the sequence information of the PKIB (SEQ ID NO 1; GenBank accession number: NM_{—}181795) or NAALADL2 (SEQ ID NO 3; GenBank accession number: NM_{—}207015 or SEQ ID NO 5; GenBank accession number: AK021754). For example, the cDNA of PKIB or NAALADL2 may be used as the probes. If necessary, the probe may be labeled with a suitable label, such as dyes, fluorescent and isotopes, and the expression level of the gene may be detected as the intensity of the hybridized labels. Furthermore, the transcription product of PKIB or NAALADL2 may be quantified using primers by amplification-based detection methods (e.g., RT-PCR). Such primers can also be prepared based on the available sequence information of the gene. For example, the primers (SEQ ID NO 8 to 15) used in the Example may be employed for the detection by RT-PCR or Northern blot, but the present invention is not restricted thereto.

Specifically, a probe or primer used for the present method hybridizes under stringent, moderately stringent, or low stringent conditions to the mRNA of PKIB or NAALADL2. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5° C. lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes or primers (e.g., 10 to 50 nucleotides) and at least about 60° C. for longer probes or primers. Stringent conditions may also be achieved with the addition of destabilizing agents, such as formamide.

Alternatively, the translation product may be detected for the diagnosis of the present invention. For example, the quantity of PKIB or NAALADL2 protein may be determined. A method for determining the quantity of the protein as the translation product includes immunoassay methods that use an antibody specifically recognizing the PKIB or NAALADL2 protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to PKIB or NAALADL2 protein. Methods to prepare these kinds of antibodies for the detection of proteins are well known in the art, and any method may be employed in the present invention to prepare such antibodies and equivalents thereof. Preferably, the antibody binding to PKIB is prepared with epitope peptide comprising amino acid sequence of SARAGRRNALPDIQSSAATD (SEQ ID NO: 33) or KEKDEKTTQDQLEKPQNEEK (SEQ ID NO: 34). Whereas the antibody binding to NAALADL2 is prepared with epitope peptide comprising extracellular domain (SEQ ID NO: 32).

As another method to detect the expression level of PKIB or NAALADL2 gene based on its translation product, the intensity of staining may be observed via immunohistochemical analysis using an antibody against PKIB or NAALADL2 protein. Namely, the observation of strong staining indicates increased presence of the protein and at the same time high expression level of PKIB or NAALADL2 gene.

Moreover, in addition to the expression level of PKIB or NAALADL2 gene, the expression level of other cancer-associated genes, for example, genes known to be differentially expressed in prostate cancer may also be determined to improve the accuracy of the diagnosis.

The expression level of PKIB or NAALADL2 gene in a biological sample can be considered to be increased if it increases from the control level of the PKIB or NAALADL2 gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of PKIB or NAALADL2 gene in samples from subjects whose disease state are known. Furthermore, the control level can be a database of expression patterns from previously tested cells. Moreover, according to an aspect of the present invention, the expression level of PKIB or NAALADL2 gene in a biological sample may be compared to multiple control levels, which control levels are determined from multiple reference samples. It is preferred to use a control level determined from a reference sample derived from a tissue type similar to that of the patient-derived biological sample. Moreover, it is preferred, to use the standard value of the expression levels of PKIB or NAALADL2 gene in a population with a known disease state. The standard value may be obtained by any method known in the art. For example, a range of mean±2 S.D. or mean±3 S.D. may be used as standard value. In the context of the present invention, a control level determined from a biological sample that is known not to be cancerous is called “nomial control level”. On the other hand, if the control level is determined from a cancerous biological sample, it will be called “cancerous control level”.

When the expression level of PKIB or NAALADL2 gene is increased compared to the normal control level or is similar to the cancerous control level, the subject may be diagnosed to be suffering from or at a risk of developing cancer. Furthermore, in case where the expression levels of multiple cancer-related genes are compared, a similarity in the gene expression pattern between the sample and the reference which is cancerous indicates that the subject is suffering from or at a risk of developing cancer.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

### Screening for Anti-Prostate Cancer Compound

In the context of the present invention, agents to be identified through the present screening methods may be any compound or composition including several compounds. Furthermore, the test agent exposed to a cell or protein according to the screening methods of the present invention may be a single compound or a combination of compounds. When a combination of compounds is used in the methods, the compounds may be contacted sequentially or simultaneously.

Any test agent, for example, cell extracts, cell culture supernatant, products of fermenting microorganism, extracts from marine organism, plant extracts, purified or crude proteins, peptides, non-peptide compounds, synthetic micromolecular compounds (including nucleic acid constructs, such as antisense RNA, siRNA, ribozymes, etc.) and natural compounds can be used in the screening methods of the present invention. The test agent of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound screened by any of the present screening methods is converted by addition, deletion and/or replacement, is included in the agents identified by the screening methods of the present invention.

Furthermore, when the screened test agent is a protein, for obtaining a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA is confirmed by its usefulness in preparing the test agent which is a candidate for treating or preventing cancer.

Test agents useful in the screenings described herein can also be antibodies that specifically bind to PKIB or NAALADL2 protein or partial peptides thereof that lack the biological activity of the original proteins in vivo.

Although the construction of test agent libraries is well known in the art, herein below, additional guidance in identifying test agents and construction of libraries of such agents for the present screening methods are provided.

### (i) Molecular Modeling

Construction of test agent libraries is facilitated by knowledge of the molecular structure of compounds known to have the properties sought, and/or the molecular structure of the target molecules to be inhibited, i.e., PKIB and NAALADL2. One approach to preliminary screening of test agents suitable for further evaluation is computer modeling of the interaction between the test agent and its target.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., J Med Chem 1988, 31: 722-9; Meng et al., J Computer Chem 1992, 13: 505-24; Meng et al., Proteins 1993, 17: 266-78; Shoichet et al., Science 1993, 259: 1445-50.

### (ii) Combinatorial Chemical Synthesis

Combinatorial libraries of test agents may be produced as part of a rational drug design program involving knowledge of core structures existing in known compounds. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

(iii) Phage Display

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, M A, 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### Screening for the PKIB or NAALADL2 Binding Compound

In present invention, over-expression of PKIB or NAALADL2 was detected in prostate cancer in spite of no expression in normal organs (FIGS. 1 and 2). Therefore, using the PKIB or NAALADL2 gene, proteins encoded by the gene or transcriptional regulatory region of the gene, compounds can be screened that alter the expression of the gene or the biological activity of a polypeptide encoded by the gene. Such compounds are used as pharmaceuticals for treating or preventing prostate cancer.

The present invention provides a method of screening for an agent that binds to PKIB or NAALADL2. Because of expression of PKIB and NAALADL2 in prostate cancer, an agent that binds to PKIB or NAALADL2 is useful to suppress the proliferation of prostate cancer cells, and thus is useful for treating or preventing prostate cancer. Therefore, the present invention also provides a method for screening an agent that suppresses the proliferation of prostate cancer cells, and a method for screening an agent for treating or preventing prostate cancer using the PKIB or NAALADL2 polypeptide. Specially, an embodiment of this screening method comprises the steps of:
a) contacting a test compound with a polypeptide encoded by a polynucleotide of PKIB or NAALADL2;
b) detecting the binding activity between the polypeptide and the test compound; and
c) selecting the test compound that binds to the polypeptide.

The method of the present invention will be described in more detail below.

The PKIB or NAALADL2 polypeptide to be used for screening may be a recombinant polypeptide or a protein derived from the nature or a partial peptide thereof. The polypeptide to be contacted with a test compound can be, for example, a purified polypeptide, a soluble protein, a form bound to a carrier or a fusion protein fused with other polypeptides.

As a method of screening for proteins, for example, that bind to the PKIB or NAALADL2 polypeptide using the PKIB or NAALADL2 polypeptide, many methods well known by a person skilled in the art can be used. Such a screening can be conducted by, for example, immunoprecipitation method (e.g., following “Interaction between PKIB and PKA-C” in [Example 1]), specifically, in the following manner. The gene encoding the PKIB or NAALADL2 polypeptide is expressed in host (e.g., animal) cells by inserting the gene into an expression vector for foreign genes, such as pSV2neo, pcDNA I, pcDNA3.1, pCAGGS and pCD8. The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-α promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SRoc promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946 (1989)), the HSV TK promoter and so on. The introduction of the gene into host cells to express a foreign gene can be performed according to any method well known to those of skill in the art, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and on the like. The polypeptide encoded by PKIB or NAALADL2 gene can be expressed as a fusion protein comprising a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, and green fluorescence protein (GFP) by the use of its multiple cloning sites are commercially available. Also, a fusion protein prepared by introducing only small epitopes consisting of several to a dozen amino acids so as not to change the property of the PKIB or NAALADL2 polypeptide by the fusion can also be used. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the PKIB or NAALADL2 polypeptide (Experimental Medicine 13: 85-90 (1995)).

In immunoprecipitation, an immune complex is formed by adding these antibodies to cell lysate prepared using an appropriate detergent. The immune complex consists of the PKIB or NAALADL2 polypeptide, a polypeptide comprising the binding ability with the polypeptide, and an antibody. Immunoprecipitation can be also conducted using antibodies against the PKIB or NAALADL2 polypeptide, besides using antibodies against the above epitopes, which antibodies can be prepared as described below. An immune complex can be precipitated, for example by Protein A sepharose or Protein G sepharose when the antibody is a mouse IgG antibody. If the polypeptide encoded by PKIB or NAALADL2 gene is prepared as a fusion protein with an epitope, such as GST, an immune complex can be formed in the same manner as in the use of the antibody against the PKIB or NAALADL2 polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the PKIB or NAALADL2 polypeptide is difficult to detect by a common staining method, such as Coomassie staining or silver staining, the detection sensitivity for the protein can be improved by culturing cells in culture medium containing radioactive isotope, 35S-methionine or 35S-cystein, labeling proteins in the cells, and detecting the proteins. The target protein can be purified directly from the SDS-polyacrylamide gel and its sequence can be determined, when the molecular weight of a protein has been revealed.

As a method of screening for proteins binding to the PKIB or NAALADL2 polypeptide using the polypeptide, for example, West-Western blotting analysis (Skolnik et al., Cell 65: 83-90 (1991)) can be used. Specifically, a protein binding to the PKIB or NAALADL2 polypeptide can be obtained by preparing a cDNA library from cultured cells (e.g., LNCaP, 22Rv1, PC-3 DU-145 and C4-2B) expected to express a protein binding to the PKIB or NAALADL2 polypeptide using a phage vector (e.g., ZAP), expressing the protein on LB-agarose, fixing the protein expressed on a filter, reacting the purified and labeled PKIB or NAALADL2 polypeptide with the above filter, and detecting the plaques expressing proteins bound to the PKIB or NAALADL2 polypeptide according to the label. The polypeptide of the invention may be labeled by utilizing the binding between biotin and avidin, or by utilizing an antibody that specifically binds to the PKIB or NAALADL2 polypeptide, or a peptide or polypeptide (for example, GST) that is fused to the PKIB or NAALADL2 polypeptide. Methods using radioisotope or fluorescence and such may be also used.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, the polypeptide of the invention is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A compound binding to the polypeptide encoded by PKIB or NAALADL2 gene can also be screened using affinity chromatography. For example, the polypeptide of the invention may be immobilized on a carrier of an affinity column, and a test compound, containing a protein capable of binding to the polypeptide of the invention, is applied to the column. A test compound herein may be, for example, cell extracts, cell lysates, etc. After loading the test compound, the column is washed, and compounds bound to the polypeptide of the invention can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound compound in the present invention. When such a biosensor is used, the interaction between the polypeptide of the invention and a test compound can be observed real-time as a surface plasmon resonance signal, using only a minute amount of polypeptide and without labeling (for example, BIAcore, Pharmacia). Therefore, it is possible to evaluate the binding between the polypeptide of the invention and a test compound using a biosensor such as BIAcore.

The methods of screening for molecules that bind when the immobilized PKIB or NAALADL2 polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the PKIB or NAALADL2 protein (including agonist and antagonist) are well known to one skilled in the art.

In preferred embodiment, a test compound selected by the method of the present invention may be candidate for further screening to evaluate the therapeutic effect thereof.

### Screening for the Compound Suppressing the Biological Activity of PKIB or NAALADL2

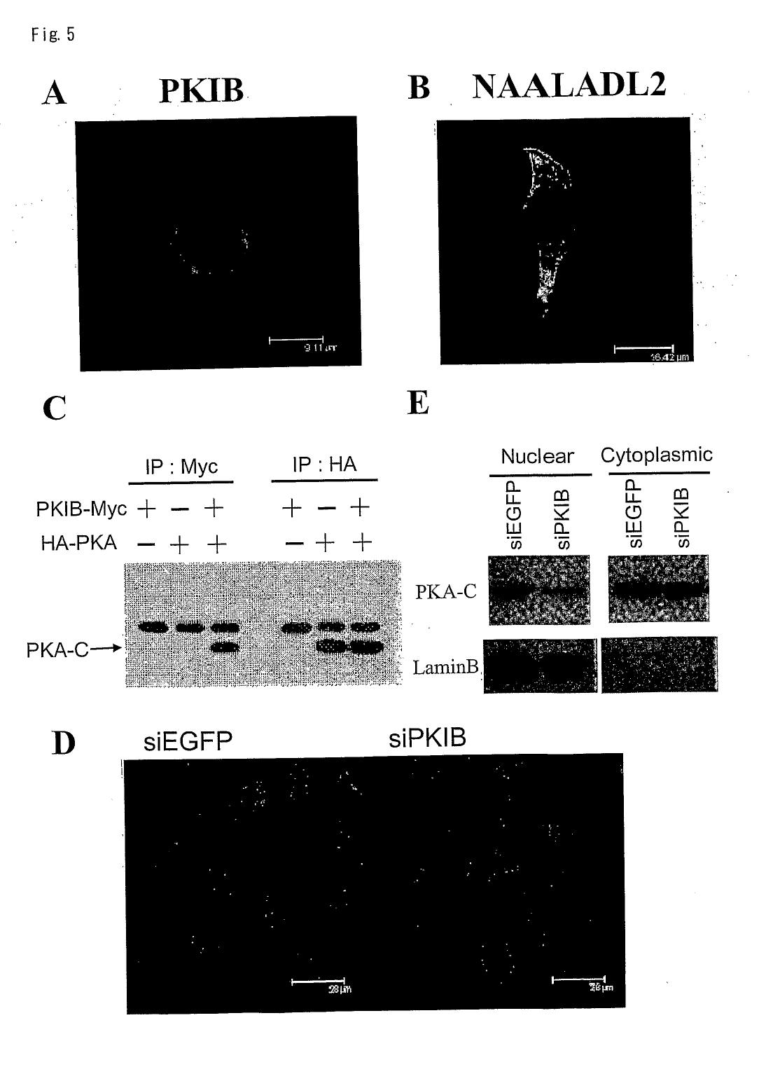

In the present invention the PKIB or NAALADL2 protein have been shown to have the activity of promoting cell proliferation of prostate cancer cells (FIGS. 3C, 4C and 6). Furthermore, PKIB have been shown to have the PKA-C nuclear accumulation activity (FIGS. 5D and E). In the present invention, the PKA-C nuclear accumulation means inhibiting export PKA-C from nuclear or accelerating PKA-C transport to nuclear. Using these biological activities, a compound which inhibits these activities of these proteins can be screened, and that compound is useful for treating or preventing prostate cancer. Therefore, the present invention also provides a method for screening a compound that suppresses the proliferation of prostate cancer cells, and a method for screening a compound for treating or preventing prostate cancer. Thus, the present invention provides a method of screening for a compound for treating or preventing prostate cancer using the polypeptide encoded by PKIB or NAALADL2 gene comprising the steps as follows:

a) contacting a test compound with a polypeptide encoded by a polynucleotide of PKIB or NAALADL2;

b) detecting the biological activity of the polypeptide of step (a); and

c) selecting the test compound that suppresses the biological activity of the polypeptide encoded by the polynucleotide of PKIB or NAALADL2 as compared to the biological activity of said polypeptide detected in the absence of the test compound.

The method of the present invention will be described in more detail below.

Any polypeptides can be used for screening so long as they comprise the biological activity of the PKIB or NAALADL2 protein. For example, PKIB or NAALADL2 protein can be used and polypeptides functionally equivalent to these proteins can also be used. Such polypeptides may be expressed endogenously or exogenously by cells. Furthermore, such biological activity includes cell-proliferating activity of the PKIB or NAALADL2 protein or PKA-C nuclear accumulation activity of the PKIB protein.

The compound isolated by this screening is a candidate for antagonists of the polypeptide encoded by PKIB or NAALADL2 gene. The term “antagonist” refers to molecules that inhibit the function of the polypeptide by binding thereto. Said term also refers to molecules that reduce or inhibit expression of the gene encoding PKIB or NAALADL2. Moreover, a compound isolated by this screening is a candidate for compounds which inhibit the in vivo interaction of the PKIB or NAALADL2 polypeptide with molecules (including DNAs and proteins).

When the biological activity to be detected in the present method is cell proliferation, it can be detected, for example, by preparing cells which express the PKIB or NAALADL2 polypeptide, culturing the cells in the presence of a test compound, and determining the speed of cell proliferation, measuring the cell cycle and such, as well as by measuring the colony forming activity, for example, shown in FIGS. 3C and 4C. “Suppress the biological activity” as defined herein are preferably at least 10% suppression of the biological activity of PKIB or NAALADL2 in comparison with in absence of the compound, more preferably at least 25%, 50% or 75% suppression and most preferably at 90% suppression.

When the biological activity to be detected in the present method is PKA-C nuclear accumulation, it can be detected, for example, by preparing cells which express the PKIB polypeptide, culturing the cells in the presence of a test compound, and determining the amount of PKA-C protein in nuclear, using immunocytochemistry or western blotting, for example, shown in FIGS. 5D and E. “Suppress the biological activity” as defined herein are preferably at least 10% suppression of the biological activity of PKIB in comparison with in absence of the compound, more preferably at least 25%, 50% or 75% suppression and most preferably at 90% suppression.

In preferred embodiment, a test compound selected by the method of the present invention may be candidate for further screening to evaluate the therapeutic effect thereof.

### Screening for the Compound Altering the Expression of PKIB or NAALADL2

In the present invention, the decrease of the expression of PKIB or NAALADL2 by siRNA has been shown to inhibit cancer cell proliferation (FIGS. 3 and 4). Therefore, the present invention provides a method of screening for an agent that inhibits the expression of PKIB or NAALADL2. An agent that inhibits the expression of PKIB or NAALADL2 is useful to suppress the proliferation of prostate cancer cells, and thus is useful for treating or preventing prostate cancer. Therefore, the present invention also provides a method for screening an agent that suppresses the proliferation of prostate cancer cells, and a method for screening an agent for treating or preventing prostate cancer. In the context of the present invention, such screening may comprise, for example, the following steps:
a) contacting a candidate compound with a cell expressing PKIB or NAALADL2; and
b) selecting the candidate compound that reduces the expression level of PKIB or NAALADL2 as compared to a control.

The method of the present invention will be described in more detail below.

Cells expressing the PKIB or NAALADL2 include, for example, cell lines established from prostate cancer; such cells can be used for the above screening of the present invention (e.g., LNCaP, 22Rv1, PC-3, DU-145 and C4-2B). The expression level can be estimated by methods well known to one skilled in the art, for example, RT-PCR, Northern bolt assay, Western bolt assay, immunostaining and flow cytometry analysis. “reduce the expression level” as defined herein are preferably at least 10% reduction of expression level of PKIB or NAALADL2 in comparison to the expression level in absence of the compound, more preferably at least 25%, 50% or 75% reduced level and most preferably at 95% reduced level. The compound herein includes chemical compound, double-strand nucleotide, and so on. The preparation of the double-strand nucleotide is in aforementioned description. In the method of screening, a compound that reduces the expression level of PKIB or NAALADL2 can be selected as candidate agents to be used for the treatment or prevention of prostate cancer.

Alternatively, the screening method of the present invention may comprise the following steps:
a) contacting a candidate compound with a cell into which a vector, comprising the transcriptional regulatory region of PKIB or NAALADL2 and a reporter gene that is expressed under the control of the transcriptional regulatory region, has been introduced;
b) measuring the expression or activity of said reporter gene; and
c) selecting the candidate compound that reduces the expression or activity of said reporter gene.

Suitable reporter genes and host cells are well known in the art. For example, reporter genes are luciferase, green fluorescence protein (GFP), Discosoma sp. Red Fluorescent Protein (DsRed), Chrolamphenicol Acetyltransferase (CAT), lacZ and β-glucuronidase (GUS), and host cell is COST, HEK293, HeLa and so on. The reporter construct required for the screening can be prepared by connecting reporter gene sequence to the transcriptional regulatory region of PKIB or NAALADL2. The transcriptional regulatory region of PKIB or NAALADL2 herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. Methods for identifying a transcriptional regulatory region, and also assay protocol are well known (Molecular Cloning third edition chapter 17, 2001, Cold Springs Harbor Laboratory Press). The vector containing the said reporter construct is infected to host cells and the expression or activity of the reporter gene is detected by method well known in the art (e.g., using luminometer, absorption spectrometer, flow cytometer and so on). “reduces the expression or activity” as defined herein are preferably at least 10% reduction of the expression or activity of the reporter gene in comparison with in absence of the compound, more preferably at least 25%, 50% or 75% reduction and most preferably at 95% reduction.

In preferred embodiment, a test compound selected by the method of the present invention may be candidate for further screening to evaluate the therapeutic effect thereof.

### Screening for the Compound Decreasing the Binding Between PKIB and PKA-C

In the present invention, the interaction between PKIB and PKA-C is shown by immunoprecipitation (FIG. 5C). Moreover, PKA-C is exported from the nucleus to the cytoplasm in the absence of PKIB (FIG. 5D). The present invention provides a method of screening for a compound that inhibits the binding between PKIB and PKA-C. A compound that inhibits the binding between PKIB and PKA-C is useful to suppress the proliferation of prostate cancer cells, and thus is useful for treating or preventing prostate cancer. Therefore, the present invention also provides a method for screening a compound that suppresses the proliferation of prostate cancer cells, and a method for screening a compound for treating or preventing prostate cancer.

More specifically, the method includes the steps of:
a) contacting a PKIB polypeptide or functional equivalent thereof with PKA-C polypeptide or functional equivalent thereof in the presence of a test compound;
b) detecting the binding between the polypeptides; and
c) selecting the test compound that inhibits the binding between the polypeptides.

“functional equivalent of PKIB polypeptide” herein refers to the polypeptide which comprises amino acid sequence of PKA-C binding domain; pseudo substrate motif (RRNA: SEQ ID NO: 31). Similarly, “functional equivalent of PKA-C polypeptide” refers to the polypeptide which comprises amino acid sequence of PKIB binding domain.

The method of the present invention will be described in more detail below.

As a method of screening for compounds that inhibit binding between PKIB and PKA-C, many methods well known by one skilled in the art can be used. Such a screening can be carried out as an in vitro assay system. More specifically, first, PKA-C or PKIB polypeptide is bound to a support, and the other polypeptide is added together with a test compound thereto. Next, the mixture is incubated, washed and the other polypeptide bound to the support is detected and/or measured. Promising candidate compound can reduce the amount of detecting the other polypeptide. Here, PKA-C or PKIB polypeptide can be prepared not only as a natural protein but also as a recombinant protein prepared by the gene recombination technique. The natural protein can be prepared, for example, by affinity chromatography. On the other hand, the recombinant protein may be prepared by culturing cells transformed with DNA encoding the PKA-C to express the protein therein and then recovering it.

Examples of supports that may be used for binding proteins include insoluble polysaccharides, such as agarose, cellulose and dextran; and synthetic resins, such as polyacrylamide, polystyrene and silicon; preferably commercial available beads and plates (e.g., multi-well plates, biosensor chip, etc.) prepared from the above materials may be used. When using beads, they may be filled into a column. Alternatively, the use of magnetic beads of also known in the art, and enables to readily isolate proteins bound on the beads via magnetism.

The binding of a protein to a support may be conducted according to routine methods, such as chemical bonding and physical adsorption. Alternatively, a protein may be bound to a support via antibodies specifically recognizing the protein. Moreover, binding of a protein to a support can be also conducted by means of avidin and biotin. The binding between proteins is carried out in buffer, for example, but are not limited to, phosphate buffer and Tris buffer, as long as the buffer does not inhibit binding between the proteins.

In the present invention, a biosensor using the surface plasmon resonance phenomenon may be used as a mean for detecting or quantifying the bound protein. When such a biosensor is used, the interaction between the proteins can be observed real-time as a surface plasmon resonance signal, using only a minute amount of polypeptide and without labeling (for example, BIAcore, Pharmacia). Therefore, it is possible to evaluate binding between PKA-C and PKIB using a biosensor such as BIAcore.

Alternatively, PKA-C or PKIB may be labeled, and the label of the polypeptide may be used to detect or measure the binding activity. Specifically, after pre-labeling one of the polypeptide, the labeled polypeptide is contacted with the other polypeptide in the presence of a test compound, and then bound polypeptide are detected or measured according to the label after washing. Labeling substances such as radioisotope (e.g., 3H, 14C, 32P, 33P, 35S, 125I, 131I), enzymes (e.g., alkaline phosphatase, horseradish peroxidase, b-galactosidase, b-glucosidase), fluorescent substances (e.g., fluorescein isothiocyanate (FITC), rhodamine) and biotin/avidin, may be used for the labeling of a protein in the present method. When the protein is labeled with radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Furthermore, binding between PKA-C and PKIB can be also detected or measured using antibodies to PKA-C or HUB. For example, after contacting PKA-C polypeptide immobilized on a support with a test compound and PKIB, the mixture is incubated and washed, and detection or measurement can be conducted using an antibody against PKIB. Alternatively, PKIB may be immobilized on a support, and an antibody against PKA-C may be used as the antibody. In case of using an antibody in the present screening, the antibody is preferably labeled with one of the labeling substances mentioned above, and detected or measured based on the labeling substance. Alternatively, the antibody against PKA-C or PKIB may be used as a primary antibody to be detected with a secondary antibody that is labeled with a labeling substance. Furthermore, the antibody bound to the protein in the screening of the present invention may be detected or measured using protein G or protein A column.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, for example, PKA-C polypeptide is fused to the SRF-binding region or GAL4-binding region and expressed in yeast cells. PKIB polypeptide that binds to PKA-C polypeptide is fused to the VP 16 or GAL4 transcriptional activation region and also expressed in the yeast cells in the existence of a test compound. Alternatively, PKIB polypeptide may be fused to the SRF-binding region or GAL4-binding region, and PKA-C polypeptide to the VP16 or GAL4 transcriptional activation region. The binding of the two activates a reporter gene, making positive clones detectable. As a reporter gene, for example, Ade2 gene, lacZ gene, CAT gene, luciferase gene and such can be used besides HIS3 gene.

Moreover, the screening method of this invention is detecting the nuclear localization of PKA-C, since PKA-C is located in the nucleus in the presence of PKIB but in the cytoplasm in the absence of PKIB. For example, the both of PKA-C and PKIB expressing cells are contacted with the test compound and washed. The cells are fixed for example, by 70% ethanol or 4% paraformaldehyde and detected with anti-PKA-C antibody. When PKA-C is approximately detected in cytoplasm, the test compound is used for preventing prostate cancer. Here, PKA-C and PKIB may be force-expressed to the cells by the method well known in the art. Accordingly, the both of PKA-C and PKIB expressing cells are contacted with the test compound and the nuclear extract of the cells is prepared by well known method (e.g., using Subcellular ProteoExtract Kit (S-PEK) produced by Merck Bioscience). The amount of PKA-C in the nuclear extract is detected by western blot assay.

In preferred embodiment, a test compound selected by the method of the present invention may be candidate for further screening to evaluate the therapeutic effect thereof. That is, the above screening method further comprises the following steps;
d) contacting the candidate compound selected in step c) with a cell expressing PKIB and PKA-C; and
e) selecting the candidate compound that reduces the phosphorylation level of Akt in comparison with the expression level detected in the absence of the candidate compound.

The detection method of Akt phosphorylation well known by one skilled in the art can be used. For example, western blot analysis described in following EXAMPLES section can be used. More preferably, the phosphorylation level of Akt (SEQ ID NO: 35) is detected at the 473 serine residue.

### Screening for the Compound Decreasing the Akt Phosphorylation Through the Inhibition of PKIB Function

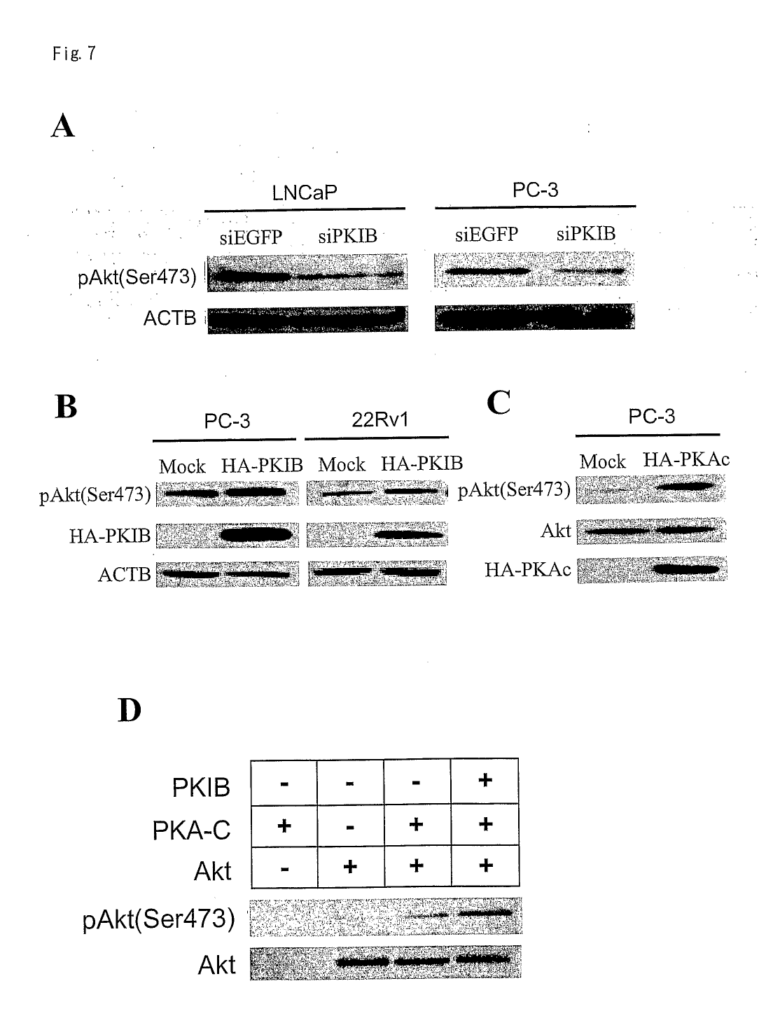

In the present invention, the Akt phosphorylation is reduced by PKIB siRNA (FIG. 7A). Moreover, the Akt phosphorylation is enhanced by both or either of PKIB and PKA-C overexpression (FIGS. 7B and C). The present invention provides a method of screening for a compound that inhibits the binding between PKIB and PKA-C. The Akt phosphorylation is likely to play critical roles in HRPC progression and its malignant phenotype (Sellers, W. R. & Sawyers, C. L. (2002) in Somatic Genetics of Prostate Cancer: Oncogenes and Tumor Suppressors ed. Kantoff, P. (Lippincott Williams & Wilkins, Philadelphia), Wang Y, Kreisberg J I, Ghosh P M., Curr Cancer Drug Targets. 2007 September; 7(6):591-604, Lin H K, Yeh S, Kang H Y, Chang C, Proc Natl Acad Sci USA 2001; 98(13):7200-5, Feldman B J, Feldman D, Nat Rev Cancer 2001; 1(1):34-45, Malik S N, Brattain M, Ghosh P M, Troyer D A, Prihoda T, Bedolla R, Kreisberg J I., Clin Cancer Res. 2002; 8(4):1168-71), so compound that decrease the Akt phosphorylation through the inhibition of PKIB function is expected to suppress the proliferation of prostate cancer cells, and thus is useful for treating or preventing prostate cancer. Therefore, the present invention also provides a method for screening a compound that suppresses the proliferation of prostate cancer cells, and a method for screening a compound for treating or preventing prostate cancer, preferably HRPC.

More specifically, the method includes the steps of:
a) contacting a candidate compound with a cell expressing PKIB and PKA-C; and
b) selecting the candidate compound that reduces the phosphorylation level of Akt in comparison with the expression level detected in the absence of the candidate compound.

Alternatively, a candidate compound suitable for the treatment and/or prevention of prostate cancer may be identifyed by the present invention. Such methods including the steps of:
(a) incubating PKIB or functionally equivalent thereof and Akt with PKA-C or functionally equivalent thereof in the presence of a test compound under conditions suitable for the phosphorylation of Akt by PKIB, wherein the Akt is a polypeptide selected from the group consisting of:
   i. a polypeptide comprising the amino acid sequence of SEQ ID NO: 35 (Akt);
   ii. a polypeptide comprising the amino acid sequence of SEQ ID NO: 35 wherein one or more amino acids are substituted, deleted, or inserted, provided said polypeptide has a biological activity equivalent to the polypeptide consisting of the amino acid sequence of SEQ ID NO: 35;
   iii. a polypeptide encoded by a polynucleotide that hybridizes under stringent conditions to a polynucleotide consisting of the nucleotide sequence of SEQ ID NO: 36, provided the polypeptide has a biological activity equivalent to a polypeptide consisting of the amino acid sequence of SEQ ID NO: 35;
(b) detecting a phosphorylation level of the Akt,
(c) comparing the phosphorylation level of the Akt measured in step (b) to a control level, and
(d) selecting a compound that decreases the phosphorylation level of the Akt as compared to the control level.

In preferred embodiment, a test compound selected by the method of the present invention may be candidate for further screening to evaluate the therapeutic effect thereof.

Preferably, the phosphorylation level of Akt may be detected at the 473 serine residue of the amino acid sequence of SEQ ID NO: 35, or homologous positions of the polypeptide. The detection method of Akt phosphorylation well known by one skilled in the art can be used. For example, western blot analysis described in following EXAMPLES section can be used.

In the context of the present invention, the conditions suitable for the phosphorylation of Akt by PKIB may be provided with an incubation of Akt and PKIB with PKA-C in the presence of phosphate donor, e.g. ATP. The conditions suitable for the Akt phosphorylation by PKIB also include culturing cells expressing PKIB, PKA-C and the polypeptides. For example, the cell may be a transformant cell harboring an expression vector containing a polynucleotide that encodes the polypeptide. After the incubation, the phosphorylation level of the Akt can be detected with an antibody recognizing phosphorylated Akt.

Prior to the detection of phosphorylated Akt, Akt may be separated from other elements, or cell lysate of Akt expressing cells. For instance, gel electrophoresis may be used for the separation of Akt from remaining components. Alternatively, Akt may be captured by contacting Akt with a carrier having an anti-Akt antibody. When the labeled phosphate donor is used, the phosphorylation level of the Akt can be detected by tracing the label. For example, when radio-labeled ATP (e.g. ³²P-ATP) is used as a phosphate donor, radio activity of the separated Akt correlates with the phosphorylation level of the Akt. Alternatively, an antibody specifically recognizing phosphorylated Akt from unphosphorylated Akt may be used to detect phosphorylated Akt. Preferably, the antibody recognizes phosphorylated Akt at Ser-473 residues.

Methods for preparing polypeptides functionally equivalent to a given protein are well known by a person skilled in the art and include known methods of introducing mutations into the protein. Generally, it is known that modifications of one or more amino acid in a protein do not influence the function of the protein (Mark D F et al., Proc Natl Acad Sci USA 1984, 81: 5662-6; Zoller M J & Smith M, Nucleic Acids Res 1982, 10: 6487-500; Wang A et al., Science 1984, 224:1431-3; Dalbadie-McFarland G et al., Proc Natl Acad Sci USA 1982, 79: 6409-13). In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids, or those considered to be “conservative modifications”, wherein the alteration of a protein results in a protein with similar functions, are contemplated in the context of the instant invention.

For example, one skilled in the art can prepare polypeptides functionally equivalent to Akt, PKA-C or PKIB by introducing an appropriate mutation in the amino acid sequence of either of these proteins using, for example, site-directed mutagenesis (Hashimoto-Gotoh et al., Gene 152:271-5 (1995); Zoller and Smith, Methods Enzymol 100: 468-500 (1983); Kramer et al., Nucleic Acids Res. 12:9441-56 (1984); Kramer and Fritz, Methods Enzymol 154: 350-67 (1987); Kunkel, Proc Natl Acad Sci USA 82: 488-92 (1985); Kunkel T A, et al., Methods Enzymol. 1991; 204:125-39.). The polypeptides of the present invention includes those having the amino acid sequences of Akt, PKAc or PKIB in which one or more amino acids are mutated, provided the resulting mutated polypeptides are functionally equivalent to Akt, PKA-C or PKIB, respectively. So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, typically 20 amino acids or less, more typically 10 amino acids or less, preferably 5-6 amino acids or less, and more preferably 1-3 amino acids.

The amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Note, the parenthetic letters indicate the one-letter codes of amino acids. Furthermore, conservative substitution tables providing functionally similar amino acids are well known in the art. For example, the following eight groups each contain amino acids that are conservative substitutions for one another:
1) Alanine (A), Glycine (G);
2) Aspartic acid (D), Glutamic acid (E);
3) Aspargine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins 1984).

Such conservatively modified polypeptides are included in the present Akt, PKA-C or PKIB protein. However, the present invention is not restricted thereto and the Akt, PKA-C and PKIB proteins include non-conservative modifications so long as the binding activity of the original proteins is retained. Furthermore, the modified proteins do not exclude polymorphic variants, interspecies homologues, and those encoded by alleles of these proteins.

An example of a polypeptide to which one or more amino acids residues are added to the amino acid sequence of Akt, PKA-C or PKIB is a fusion protein containing Akt, PKA-C or PKIB, respectively. Accordingly, fusion proteins, i.e., fusions of Akt, PKA-C or PKIB and other peptides or proteins, are included in the present invention. Fusion proteins can be made by techniques well known to a person skilled in the art, such as by linking the DNA encoding Akt, PKA-C or PKIB with DNA encoding other peptides or proteins, so that the frames match, inserting the fusion DNA into an expression vector and expressing it in a host. There is no restriction as to the peptides or proteins fused to the protein of the present invention.

Known peptides that can be used as peptides to be fused to the Akt, PKA-C or PKIB proteins include, for example, FLAG (Hopp T P et al., Biotechnology 1988 6: 1204-10), 6×His containing six His (histidine) residues, 10×His, Influenza agglutinin (HA), human c-myc fragment, VSP-GP fragment, p18HIV fragment, T7-tag, HSV-tag, E-tag, SV40T antigen fragment, lck tag, alpha-tubulin fragment, B-tag, Protein C fragment, and the like. Examples of proteins that may be fused to a protein of the invention include GST (glutathione-S-transferase), Influenza agglutinin (HA), immunoglobulin constant region, beta-alactosidase, MBP (maltose-binding protein), and such.

Fusion proteins can be prepared by fusing commercially available DNA, encoding the fusion peptides or proteins discussed above, with the DNA encoding the Akt, PKA-C or PKIB proteins and expressing the fused DNA prepared.

An alternative method known in the art to isolate functionally equivalent polypeptides involves, for example, hybridization techniques (Sambrook et al., Molecular Cloning 2nd ed. 9.47-9.58, Cold Spring Harbor Lab. Press (1989)). One skilled in the art can readily isolate a DNA having high homology with Akt (i.e., SEQ ID NO: 36), PKA-C or PKIB, and isolate polypeptides functionally equivalent to the Akt, PKA-C or PKIB from the isolated DNA. The proteins of the present invention include those that are encoded by DNA that hybridize with a whole or part of the DNA sequence encoding Akt, PKA-C or PKIB and are functionally equivalent to Akt, PKA-C or PKIB. These polypeptides include mammalian homologues corresponding to the protein derived from humans (for example, a polypeptide encoded by a monkey, rat, rabbit and bovine gene). In isolating a cDNA highly homologous to the DNA encoding Akt, PKA-C or PKIB from animals, it is particularly preferable to use prostate cancer tissues.

The condition of hybridization for isolating a DNA encoding a protein functional equivalent to the human Akt, PKA-C or PKIB protein can be routinely selected by a person skilled in the art. The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). In the context of the present invention, suitable hybridization conditions can be routinely selected by a person skilled in the art.

Generally, stringent conditions are selected to be about 5-10° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is preferably at least two times of background, more preferably 10 times of background hybridization.

Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 50° C. Suitable hybridization conditions may also include prehybridization at 68° C. for 30 min or longer using “Rapid-hyb buffer” (Amersham LIFE SCIENCE), adding a labeled probe, and warming at 68° C. for 1 h or longer.

The washing step can be conducted, for example, under conditions of low stringency. Thus, an exemplary low stringency condition may include, for example, 42° C., 2×SSC, 0.1% SDS, or preferably 50° C., 2×SSC, 0.1% SDS. Alternatively, an exemplary high stringency condition may include, for example, washing 3 times in 2×SSC, 0.01% SDS at room temperature for 20 min, then washing 3 times in 1×SSC, 0.1% SDS at 37° C. for 20 min, and washing twice in 1×SSC, 0.1% SDS at 50° C. for 20 min. However, several factors such as temperature and salt concentration can influence the stringency of hybridization and one skilled in the art can suitably select the factors to achieve the requisite stringency.

Preferably, the functionally equivalent polypeptide has an amino acid sequence with at least about 80% homology (also referred to as sequence identity) to the native Akt, PKA-C or PKIB sequence disclosed here, more preferably at least about 85%, 90%, 95%, 96%, 97%, 98%, or 99% homology. The homology of a polypeptide can be determined by following the algorithm in “Wilbur and Lipman, Proc Natl Acad Sci USA 80: 726-30 (1983)”. In other embodiments, the functional equivalent polypeptide can be encoded by a polynucleotide that hybridizes under stringent conditions (as defined below) to a polynucleotide encoding such a functional equivalent polypeptide.

In place of hybridization, a gene amplification method, for example, the polymerase chain reaction (PCR) method, can be utilized to isolate a DNA encoding a polypeptide functionally equivalent to Akt, PKA-C or PKIB, using a primer synthesized based on the sequence information for Akt or PKIB.

An Akt, PKA-C or PKIB functional equivalent useful in the context of the present invention may have variations in amino acid sequence, molecular weight, isoelectric point, the presence or absence of sugar chains, or form, depending on the cell or host used to produce it or the purification method utilized. Nevertheless, so long as it is a function equivalent of either the Akt, PKA-C or PKIB polypeptide, it is within the scope of the present invention.

### Screening for the Antibody Binding to NAALADL2

NAALADL2 is a novel type II membrane protein and belongs to glutamate carboxypeptidase II (GCPII) family. The famous prostate cancer marker, prostate-specific membrane antigen (PSMA) also belongs to GCPII family (Rajasekaran A K et al., Am J Physiol Cell Physiol 2005 288: C975-81, and Murphy G P et al., Prostate 2000 42: 145-9.). NAALADL2 shows homology with PSMA and has one transmembrane and localized at the plasma membrane (FIG. 5B). PSMA is the target of a FDA-approved prostate cancer-imaging agent, the ¹¹¹In-labeled 7E11 monoclonal antibody (Prostascint, Cytogen, Princeton, N.J.), and PSMA is targeted by monoclonal antibodies such as J591, which is in clinical trials for specific delivery of imaging agent or therapeutics to PSMA-expressing cells (Murphy G P et al., Prostate 2000 42: 145-9, and Holmes E H, Expert Opin Investig Drugs 2001 10: 511-9.). Therefore, anti-NAALADL2 antibody that may be used in diagnosing or preventing prostate cancer can be identified through screenings that use the binding ability of NAALADL2 in cell surface as indices. An embodiment of this screening method comprises the step of:
a) contacting a candidate antibody with a cell expressing NAALADL2;
b) selecting the test antibody that binds to NAALADL2 on the cell surface.

The method of the present invention will be described in more detail below.

Alternatively, putative extracelluar domain of NAALADL2 is SEQ ID NO: 32. Therefore, the method of screening the antibody binding to NAALADL2 at extracelluar comprised the step of;
a) contacting a candidate antibody with the polypeptide consisting of SEQ ID NO: 32;
b) selecting the test antibody that binds to the polypeptide.

The antibody or antibody fragment or non-antibody binding protein which is described in following section is selected by detecting affinity of NAALADL2 expressing cells like prostate cancer cell. Unspecific binding to these cells is blocked by treatment with PBS containing 3% BSA for 30 min at room temperature. Cells are incubated for 60 min at room temperature with candidate antibody or antibody fragment. After washing with PBS, the cells are stained by FITC-conjugated secondary antibody for 60 min at room temperature and detected by using fluorometer. Alternatively, a biosensor using the surface plasmon resonance phenomenon may be used as a mean for detecting or quantifying the antibody or antibody fragment in the present invention. The antibody or antibody fragment which can detect the NAALADL2 peptide on the cell surface is selected in the presence invention.

In preferred embodiment, a test compound selected by the method of the present invention may be candidate for further screening to evaluate the therapeutic effect thereof.

### Antibody

The terms “antibody” as used herein is intended to include immunoglobulins and fragments thereof which are specifically reactive to the designated protein or peptide thereof. An antibody can include human antibodies, primatized antibodies, chimeric antibodies, bispecific antibodies, humanized antibodies, antibodies fused to other proteins or radiolabels, and antibody fragments. Furthermore, an antibody herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. An “antibody” indicates all classes (e.g. IgA, IgD, IgE, IgG and IgM).

The subject invention uses antibodies to HUB or NAALADL2. More preferably, the antibodies to a protein comprising amino acid sequence of SEQ ID NO: 33 or 34 can be used as the antibodies to HUB, and the antibodies to a protein comprising amino acid sequence of SEQ ID NO: 32 can be used as the antibodies to NAALADL2. These antibodies will be provided by known methods. Exemplary techniques for the production of the antibodies used in accordance with the present invention are described.

### (i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R′N═C═NR, where R′ and R are different alkyl groups.

Animals are immunized against the antigen, immunogenic conjugates, or derivatives by combining, e.g. 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent.

Conjugates also can be made in recombinant cell culture as protein fusions. Also, aggregating agents such as alum are suitably used to enhance the immune response.

### (ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256: 495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133: 300 1 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107: 220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5: 256-262 (1993) and Pluckthun, Immunol. Revs., 130: 151-188 (1992).

Another method of generating specific antibodies, or antibody fragments, reactive against a PKIB or a NAALADL2 is to screen expression libraries encoding immunoglobulin genes, or portions thereof, expressed in bacteria with a PKIB or a NAALADL2 protein or peptide. More preferably, PKIB fragment comprising the amino acid sequence of SEQ ID NO: 33 or 34 can be used as a substitution for PKIB, and NAALADL2 fragment comprising the amino acid sequence of SEQ ID NO: 32 can be used as a substitution for NAALADL2. For example, complete Fab fragments, VH regions and Fv regions can be expressed in bacteria using phage expression libraries. See for example, Ward et al., Nature 341: 544-546 (1989); Huse et al., Science 246: 1275-1281 (1989); and McCafferty et al., Nature 348: 552-554 (1990). Screening such libraries with, for example, a PKIB peptide, PKIB fragment comprising the amino acid sequence of SEQ ID NO: 33 or 34, a NAALADL2 peptide or NAALADL2 fragment comprising the amino acid sequence of SEQ ID NO: 32, can identify immunoglobulin fragments reactive with PKIB, the PKIB fragment, NAALADL2 or NAALADL2 fragment. Alternatively, the SCID-hu mouse (available from Genpharm) can be used to produce antibodies or fragments thereof.

In a further embodiment, antibodies or antibody fragments can be isolated from antibody phage libraries generated using the techniques described in McCafferty et al., Nature, 348: 552-554 (1990). Clackson et al., Nature, 352: 624-628 (1991) and Marks et al., J MoL BioL, 222: 581-597 (1991) describe the isolation of murine and human antibodies, respectively, using phage libraries. Subsequent publications describe the production of high affinity (nM range) human antibodies by chain shuffling (Marks et al., Bio/Technology, 10: 779-783 (1992)), as well as combinatorial infection and in vivo recombination as a strategy for constructing very large phage libraries (Waterhouse et al., Nuc. Acids. Res., 21: 2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. ScL USA, 81: 6851 (1984)), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigencombining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321: 522-525 (1986); Reichmann et al., Nature, 332: 323-327 (1988); Verhoeyen et al., Science, 239: 1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so called“best-fit” method, the sequence of the variable domain of a rodent antibody is screened against the entire library of known human variable-domain sequences. The human sequence which is closest to that of the rodent is then accepted as the human framework region (FR) for the humanized antibody (Suns et al., J. Immunol., 151: 2296 (1993); Chothia et al., J. Mol. Biol, 196: 901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89: 4285 (1992); Presta et al., J. Immunol., 151: 2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen, is achieved. In general, the hypervariable region residues are directly and most substantially involved in influencing antigen binding.

### (iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ line mutant mice will result in the production of human antibodies upon antigen challenge. See, e.g., Jakobovits et al., Proc. Mad. Acad. Sci. USA, 90: 255 1 (1993); Jakobovits et al., Nature, 362: 255-258 (1993); Bruggermann et al., Year in immuno., 7: 33 (1993); and U.S. Pat. Nos. 5,591,669, 5,589,369 and 5,545,807.

Alternatively, phage display technology (McCafferty et al., Nature 348: 552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3: 564-57 1 (1993). Several sources of V-gene segments can be used for phage display.

Clackson et al., Nature, 352: 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self antigens) can be isolated essentially following the techniques described by Marks et al., J. Mol. Biol, 222: 581-597 (1991), or Griffith et al., EMBO J. 12: 725-734 (1993). See, also, U.S. Pat. Nos. 5,565,332 and 5,573,905.

Human antibodies may also be generated by in vitro activated B cells (see U.S. Pat. Nos. 5,567,610 and 5,229,275). A preferred means of generating human antibodies using SCID mice is disclosed in commonly-owned, co-pending applications.

### (v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24: 107-117 (1992) and Brennan et al., Science, 229: 81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F (ab′) 2 fragments (Carter et al., Bio/Technology 10: 163-167 (1992)). According to another approach, F (ab′) 2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

### (vi) Non-Antibody Binding Protein

The terms “non-antibody binding protein” or “non-antibody ligand” or “antigen binding protein” interchangeably refer to antibody mimics that use non-immunoglobulin protein scaffolds, including adnectins, avimers, single chain polypeptide binding molecules, and antibody-like binding peptidomimetics, as discussed in more detail below.

Other compounds have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light chain variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc Natl Acad Sci USA 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc Natl Acad Sci USA 96(5):1898-1903 (1999)) discloses an antibody mimic based on a lipocalin scaffold (Anticalin®). Lipocalins are composed of a β-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies.

Anticalins® are small, single chain peptides, typically between 160 and 180 residues, which provides several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric conformation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell Mol Biol. 49(2):209-216 (2003)) discusses a methodology for reducing antibodies into smaller peptidomimetics, they term “antibody like binding peptidemimetics” (ABiP) which can also be useful as an alternative to antibodies.

Silverman et al. (Nat Biotechnol. (2005), 23: 1556-1561) discloses fusion proteins that are single-chain polypeptides comprising multiple domains termed “avimers.” Developed from human extracellular receptor domains by in vitro exon shuffling and phage display the avimers are a class of binding proteins somewhat similar to antibodies in their affinities and specificities for various target molecules. The resulting multidomain proteins can comprise multiple independent binding domains that can exhibit improved affinity (in some cases sub-nanomolar) and specificity compared with single-epitope binding proteins. Additional details concerning methods of construction and use of avimers are disclosed, for example, in US Pat. App. Pub. Nos. 20040175756, 20050048512, 20050053973, 20050089932 and 20050221384.

In addition to non-immunoglobulin protein frameworks, antibody properties have also been mimicked in compounds comprising RNA molecules and unnatural oligomers (e.g., protease inhibitors, benzodiazepines, purine derivatives and beta-turn mimics) all of which are suitable for use with the present invention.

Although the construction of test agent libraries is well known in the art, herein below, additional guidance in identifying test agents and construction libraries of such agents for the present screening methods are provided.

(vii) Antibody Conjugates and Other Modifications

The antibodies used in the methods or included in the articles of manufacture herein are optionally conjugated to cytotoxic or therapeutic agent.

Conjugates of an antibody and one or more small molecule toxins, such as a calicheamicin, a maytansine (U.S. Pat. No. 5,208,020), a trichothecin, and CC 1065 are also contemplated herein. In one preferred embodiment of the invention, the antibodies is conjugated to one or more maytansine molecules (e.g. about 1 to about 10 maytansine molecules per antibodies molecule). Maytansine may, for example, be converted to May SS-Me which may be reduced to May-SH3 and reacted with modified antibodies (Chari et al. Cancer Research 52: 127-131 (1992)) to generate a maytansinoid-antibody conjugate. Alternatively, the antibody may be conjugated to one or more calicheamicin molecules. The calicheamicin family of antibiotics is capable of producing double stranded DNA breaks at sub-picomolar concentrations. Structural analogues of calicheamicin which may be used include, but are not limited to gamma₁^{I}, alpha₂^{I}, alpha₃^{I}, N-acetyl-gamma₁^{I}, PSAG and theta₁^{I} (Hinman et al. Cancer Research 53: 3336-3342 (1993) and Lode et al, Cancer Research 58: 2925-2928 (1998)).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha sarcin, Aleurites fordii proteins, dianthin proteins, Phytolacca americana proteins (PAPI, PAPII, and PAP-S), momordica charantia inhibitor, curcin, crotin, saponaria officinalis inhibitor, gelonin, mitogellin, restrictocin, phenomycin, neomycin and the trichothecenes. See, for example, WO 93/21232 published Oct. 28, 1993.

The present invention further contemplates antibody conjugated with a variety of radioactive isotopes. Examples include ²¹¹At, ¹³¹I, ¹²⁵I, ⁹⁰Y, ¹⁸⁶Re, ¹⁸⁸Re, ¹⁵³Sm, ²¹²Bi, ³²P and radioactive isotopes of Lu.

Conjugates of the antibody and cytotoxic agent may be made using a variety of bifunctional protein coupling agents such as N-succinimidyl-3-(2-pyridylthio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1 isothiocyanatobenzyl-3-methyldiethylene triaminepenta acetic acid (MX-DTPA) is an exemplary chelating agent for conjugation of radionuclide to the antibody. See WO94/11026. The linker may be a “cleavable linker” facilitating release of the cytotoxic drug in the cell. For example, an acid-labile linker, peptidase-sensitive linker, dimethyl linker or disulfide-containing linker (Charm et al. Cancer Research 52: 127-131 (1992)) may be used.

Alternatively, a fusion protein comprising the antibody and cytotoxic agent may be made, e.g. by recombinant techniques or peptide synthesis.

In yet another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionuclide).

The antibodies of the present invention may also be conjugated with a prodrug activating enzyme which converts a pro-drug (e.g. a peptidyl chemotherapeutic agent, see WO81/01145) to an active anti-cancer drug. See, for example, WO 88/07378 and U.S. Pat. No. 4,975,278.

The enzyme component of such conjugates includes any enzyme capable of acting on a prodrug in such a way so as to convert it into its more active, cytotoxic form. Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic5-fluorocytosine into the anti-cancer drug, fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydratecleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as“abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The enzymes of this invention can be covalently bound to the antibody by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984)).

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol.

The antibodies disclosed herein may also be formulated as liposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J; Biol. Chem. 257: 286-288 (1982) via a disulfide interchange reaction. A chemotherapeutic agent is optionally contained within the liposome. See Gabizon et al. J National Cancer Inst. 81 (19) 1484 (1989).

Amino acid sequence modifications of antibodies described herein are contemplated. For example, it may be desirable to improve the binding affinity and/or other biological properties of the antibody. Amino acid sequence variants of the antibody are prepared by introducing appropriate nucleotide changes into the antibody encoding nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the anitbody, such as changing the number or position of glycosylation sites.

A useful method for identification of certain residues or regions of the antibody that are preferred locations for mutagenesis is called “alanine scanning mutagenesis” as described by Cunningham and Wells Science, 244: 1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody of an enzyme, or a polypeptide which increases the serum half-life of the antibody.

Another type of variant is an amino acid substitution variant. These variants have at least one amino acid residue in the antibody molecule replaced by different residue. The sites of greatest interest for substitutional mutagenesis of antibody include the hypervariable regions, but FR alterations are also contemplated.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Naturally occurring residues are divided into groups based on common side-chain properties:

(1) hydrophobic: norleucine, met, ala, val, leu, ile;
(2) neutral hydrophiuic: cys, ser, thr;
(3) acidic: asp, glu;
(4) basic: asn, gln, his, lys, arg;
(5) residues that influence chain orientation: gly, pro; and
(6) aromatic: trp, tyr, phe.
Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bonds may be added to the antibody to improve its stability (particularly where the antibody is a fragment such as an Fv fragment).

A particularly preferred type of substitutional variant involves substituting one or more hypervariable region residues of a parent antibody (e.g. a humanized or human antibody). Generally, the resulting variants selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identified hypervariable region residues contributing significantly to antigen binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain.

Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to modify the antibodies used in the invention to improve effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176: 1191-1195 (1992) and Shopes, B. J limmunol 148: 2918-2922 (1992).

Homodimeric antibodies with enhanced anti-tumor activity may also be prepared using heterobifunctional cross-linkers as described in Wolff et al. Cancer Research 53: 2560-2565 (1993). Alternatively, an antibody can be engineered which has dual Fc regions and may thereby have enhanced complement lysis and ADCC capabilities. See Stevenson et al. Anti-CancerDrugDesign 3: 2 19-230 (1989).

To increase the serum half life of the antibody, one may incorporate a salvage receptor binding epitope into the antibody (especially an antibody fragment) as described in U.S. Pat. No. 5,739,277, for example. As used herein, the term“salvage receptor binding epitope” refers to an epitope of the Fc region of an IgG molecule (e.g., IgGI, IgG2, IgG3, or IgG4) that is responsible for increasing the in vivo serum half-life of the IgG molecule.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be further described in the following examples, which do not limit the scope of the invention described in the claims.

### EXAMPLE

The invention will be further described in the following examples, which do not limit the scope of the invention described in the claims.

### Example 1

### General Methods

### Cell Lines

COS7 cell and, PC cell lines LNCaP, 22Rv 1, PC-3, DU-145 and C4-2B were purchased from the American Type Culture Collection (ATCC, Rockville, Md.), and LNCaP-derived HRPC cell line C4-2B was purchased from ViroMed Laboratories (Minnetonka, Minn.). LNCaP that was passed more than 30 times was defined as LNCaP(HP), which was different from LNCaP cell at low passage morphologically and at their gene-expression pattern. They were grown in Delbecco's modified Eagle's medium (Invitrogen, Carlsbad, Calif.); this media were supplemented with 10% fetal bovine serum (Gemini Bio-Products, West Sacramento, Calif.) and 1% antibiotic/antimycotic solution (Sigma-Aldrich, St. Louis, Mo.). Cells were maintained at 37° C. in atmospheres of humidified air with 5% CO₂.

### Semi-Quantitative RT-PCR

Purification of PC cells and normal prostatic epithelial cells from frozen PC tissues was described previously (Tamura K et al., Cancer Res 2007 67: 5117-25.). Tissue samples were obtained with informed consent from HRPC patients undergoing prostatic needle biopsy, bone biopsy, TUR-P (transurethral resection of the prostate), and “warm” autopsy. Simultaneously, hormone-naïve prostate cancer (HNPC) samples were also obtained from untreated operable cases undergoing radical prostatectomy, and normal prostatic epithelial cells (NPEC) were also obtained from benign prostatic hyperplasia (BPH) patients and bladder cancer patients, who were confirmed no apparent prostate cancers or PINs histopathologically. Microdissection of HRPC cells, hormone-naïve prostatic cancer cells, and normal prostatic epithelial cells were described previously (Tamura K et al., Cancer Res 2007 67: 5117-25.). RNAs from these samples were subjected to two-round of T7-based RNA amplification (Epicentre Technologies, Madison, Wis.) and subsequent synthesis of single-strand cDNA. Total RNAs from human HRPC cell lines were extracted using RNeasy Kit (QIAGEN, Valencia, Calif.) according to manufacture's recommendation. Extracted RNAs were treated with RNase-Free DNase Set (QIAGEN) and reverse-transcribed to single-stranded cDNAs using d(T)12-18 primer with Superscript II reverse transcriptase (Invitrogen). The following primer sequences were used.

(SEQ ID NO. 6)
β-actin (ACTB) forward: TTGGCTTGACTCAGGATTTA
(SEQ ID NO. 7)
β-actin (ACTB) reverse: ATGCTATCACCTCCCCTGTG
(SEQ ID NO. 8)
PKIB forward: GGCACATACTAGAAGCAAAATACG
(SEQ ID NO. 9)
PKIB reverse: GATGGGCAAATCATTCTTGGTA
(SEQ ID NO. 10)
NAALADL2 forward: GAAAGCATCTCACATTGGTTTTC
(SEQ ID NO. 11)
NAALADL2 reverse: GGGTTTCAAAGAGAAACTCTGCT
(SEQ ID NO. 12)
NAALADL2-2 forward: GAAGCAAAATGCCAGATGGT
(SEQ ID NO. 13)
NAALADL2-2 reverse: TCCTGCACAGTGTTCTAGAAAGG

The connection between this EST and NAALADL2 gene was confirmed by RT-PCR. The RT-PCR exponential phase was determined to allow semi-quantitative comparisons among cDNAs developed from identical reactions. Each PCR regime involved a 98° C., 30 sec initial denaturation step followed by 22 cycles (for ACTB), 23 cycles (for PKIB, NAALADL2), and at 98° C. for 10 sec, 55° C. for 5 sec, and 72° C. for 30 sec, on a Gene Amp PCR system 9600 (PE Applied Biosystems, Foster, Calif.).

### Northern Blot Analysis

The total RNAs from seven PC cell lines were extracted by using RNeasy Kit (QIAGEN) and northern blot analysis was performed. With mRNA Purification Kit (GE Healthcare) mRNA was purified, according to the manufacturer's protocols. A 1 μg aliquot of each mRNA from PC cell lines, as well as those isolated from normal human heart, lung, liver, kidney, brain, and prostate (BD Biosciences, Palo Alto, Calif.), were separated on 1% denaturing agarose gels and transferred onto nylon membranes. The 261-bp probe specific to PKIB was prepared by PCR using the primer set described above and the 706-bp probe specific to NAALADL2 was prepared by PCR using the following primer set: forward 5′-ccagtgcccagaaaccaata-3′ (SEQ ID NO.14) and reverse 5′-tcaattcttcccatccaagaca-3′ (SEQ ID N0.15). Hybridization with a random-primed, α32P-dCTP-labeled probe was carried out according to the instructions for Megaprime DNA labeling system (GE bioscience, UK). Prehybridization, hybridization and washing were performed according to the supplier's recommendations. The blots were auto-radiographed with intensifying screens at −80° C. for 7 days.

Small Interfering RNA (siRNA)-Expressing Constructs and Transfection

To knock down endogenous PKIB and NAALADL2 expression in PC cells, the psiU6BX3.0 vector was used for expression of short hairpin RNA against a target gene as described previously (Tamura K et al., Cancer Res 2007 67: 5117-25.). The target sequences of the synthetic oligonucleotides for siRNA for PKIB were as follows:

| | |
|---|---|
| si1; GCCCTAAGCAGCATGTGTA, | (SEQ ID NO. 16) |
| si2; GCAGTAGGCACTTAAGCAT | (SEQ ID NO. 17) |
| si3; GATGCAAAAGAGAAAGATG | (SEQ ID NO. 18) |

The target sequences of the synthetic oligonucleotides for siRNA for NAALADL2 were as follows:

| | |
|---|---|
| si#690; GACTCAGTGGACCTCTTTG | (SEQ ID NO. 19) |
| si#913; GTCATCGATGTGAGTTATG | (SEQ ID NO. 20) |
| si#1328; GAGTCGTCAGCATGCAAGT | (SEQ ID NO. 21) |

and siEGFP; GAAGCAGCACGACTTCTTC (SEQ ID NO.22) (as a negative control). PC cell lines 22Rv1 and LNCaP(HP) or C4-2B cells were plated onto 10-cm dishes or 6-well plates, and transfected with plasmid designed to express siRNA to PKIB or NAALADL2 (8 μg/dish, 3 μg/well) using FuGENE6 (Roche) according to manufacture's instruction. Cells were selected by 0.8 mg/ml of Geneticin (Sigma-Aldrich) for 7 days, and then harvested to analyze knockdown effect on PKIB or NAALADL2 expression. RT-PCR for PKIB or NAALADL2 was performed by using the primers described above. For colony formation assay, transfectants expressing siRNAs were grown for 20 days in media containing Geneticin. After fixation with 100% methanol, transfected cells were stained with 0.1% of crystal violet-H20 to assess colony formation. Cell viability was quantified using Cell counting kit-8 (DOJINDO, Kumamoto, Japan). After 20-day culturing in the Geneticin-containing medium, the solution was added at a final concentration of 10%. Following incubation at 37° C. for 2 hours, absorbance at 490 nm and at 630 nm as reference, was measured with a Microplate Reader 550 (Bio-Rad, Hercules, Calif.). This inventors also synthesized RNA duplex corresponding to si#690 as 5′-GACUCAGUGGACCUCUUUGGG-3′ (SEQ ID NO.23) and 5′-CAAAGAGGUCCACUGAGUCUU-3′ (SEQ ID NO.24) or a negative control siRNA duplex (GCAGCACGACUUCTUUCAAGTT (SEQ ID NO.25) and CUUGAAGAAGUCGUGCUGCTT (SEQ ID NO.26)), and each of them was transfected into another NAALADL2-expressing PC cell line, C4-2B cells.

### Immunocytochemistry

The cDNA encoding an open reading frame of PKIB or NAALADL2 was amplified by PCR, and the PCR-amplified product was cloned into pcDNA3.1(+)/myc-HisA vector (Invitrogen) or pIRES/HA (Clontech/BD Bioscience). The plasmids were transfected into COS7, which were plated on glass coverslips (Becton Dickinson Labware, Franklin Lakes, N.J.), using FuGENE6 according to the manufacture's recommended procedures (Roche). After 48 hours incubation, cells were fixed with 4% paraformaldehyde, and permeablilized with 0.1% Triton X-100 in PBS for 1 min at room temperature. Unspecific binding was blocked by treatment with PBS containing 3% BSA for 30 min at room temperature. Cells were incubated for 60 min at room temperature with anti-Myc antibody (Santa Cruz) or anti-HA antibody (Sigma) diluted in PBS containing 1% BSA. After washing with PBS, cells were stained by FITC-conjugated secondary antibody (Santa Cruz) for 60 min at room temperature. After washing with PBS, specimen was mounted with VECTASHIELD (VECTOR Laboratories, Inc, Burlingame, Calif.) containing 4′,6′-diamidine-2′-phenylindoldihydrochloride (DAPI) and visualized with Spectral Confocal Scanning Systems (Leica, Bensheim, Germany).

### Interaction Between PKIB and PKA-C

PKIB-Myc and HA-PKA-C expression vectors were co-transfected into 22Rv 1 cells, and 48 hours after the transfection, these cells were lysed by lysis buffer [50 mM Tris-HCl (pH7.0), 250 mM sucrose, 1 mM DTT, 10 mM EDTA, 1 mM EGTA, 5 mM MgCl2]. The cell lysate was immunoprecipitated by anti-Myc antibody (Santa Cruz) or anti-HA antibody (Sigma), and these immunoprecitates were subject with western blot analysis with anti-Myc antibody or anti-HA antibody.

### PKA-C Localization

The present inventors synthesized the RNA duplex corresponding to the sequence si 1 (5′-GCCCUAAGCAGCAUGUGUAUA-3′ (SEQ ID NO.27) and 5′-UACACAUGCUGCUUAGGGCUU-3′ (SEQ ID NO.28)) to knock down endogenous PKIB more efficiently. This RNA duplex was transfected into PC-3 cells using Lipofectamin 2000 (Invitrogen) according to the manufacture's recommended procedures. After 48 hours incubation, cells were fixed with 4% paraformaldehyde, and permeablilized with 0.1% Triton X-100 in PBS for 1 min at room temperature. Unspecific binding was blocked by treatment with PBS containing 3% BSA for 30 min at room temperature. Cells were incubated for 60 min at room temperature with anti-PKA-C antibody (C-20, Santa Cruz) diluted at 1:200 by PBS containing 1% BSA. After washing with PBS, cells were stained by FITC-conjugated secondary antibody (Santa Cruz) for 60 min at room temperature, and visualized with Spectral Confocal Scanning Systems. To fractionate cell lysates, RNA duplex was transfected into LNCaP(HP) cells using Lipofectamin 2000 (Invitrogen) according to the manufacture's recommended procedures. After 48 hours incubation, cells were harvested and these cell lysates were fractionated by NE-PER Nuclear and Cytoplasmic Extraction Reagent (PIERCE). 30 ug protein of the fractionated cell lysates were western-blotted by using anti-PKA-C antibody and anti-laminB antibody (Calbiochem) for the loading and nucleus-fractionated control.

### Generation of PKIB-Overexpressing Cells and In Vitro/In Vivo Growth Assay

The cDNA encoding an open reading frame of PKIB was amplified by PCR, and the PCR-amplified product was cloned into pIRES/HA (Clontech/BD Bioscience). The plasmids were transfected into the PKIB-null PC cell line DU145 using FuGENE6 (Roche) according to the manufacture's recommended procedures. A population of cells was selected with 0.6 mg/ml Geneticin (Invitrogen), and clonal DU145 cells were sub-cloned by limiting dilution. PKIB expression was confirmed by RT-PCR described above, and three clones that expressed PKIB constitutively were established. Control DU145 cells transfected with empty pIRES/HA vector was also established as Mock cells. The growth curve of these established clones were measured by using Cell-counting kit-8 (DOJINDO).

For in vivo growth assay, 2×10⁶ cells of two stable clones and two Mock clones were inoculated to the right flank and the left flank of male nude mice, respectively.

### Antibody Generation and Immunohistochemistry

The two peptides from human PKIB (SARAGRRNALPDIQSSAATD (SEQ ID NO: 33) and KEKDEKTTQDQLEKPQNEEK (SEQ ID NO: 34)) were immunized into rabbits, and the immune sera were purified on affinity-columns packed with Affi-Gel 10 activated affinity media (Bio-Rad Laboratories, Hercules, Calif.) conjugating each of the peptide antigens with accordance of basic methodology. Conventional sections from PC tissues were obtained from surgical specimens, and HRPC tissues were obtained by autopsy and TUR-P (Tamura et al. Cancer Res 67, 5117-25, 2007). The sections were deparaffinized and autoclaved at 108° C. in Dako Cytomation Target Retrieval Solution High pH (Dako, Carpinteria, Calif.) for 15 min. After blocking of endogenous peroxidase and proteins, the sections were incubated with anti-PKIB antibody (diluted by 1:100) at room temperature for 60 min. After washing with PBS, immunodetection was performed with peroxidase labeled anti-rabbit immunoglobulin (Envision kit, Dako). Finally, the reactants were developed with 3,3′-diaminobenzidine (Dako). Counterstaining was performed using hematoxylin.

### Akt Phosphorylation

22Rv1, LNCaP or PC-3 cells transfected PKIB oligo siRNA corresponding to the sequence si1 or PKIB expression vector (pcDNA3.1/HA-PKIB) described above, and harvested after 48 hours or 24 hours. The cells were lysed with RIPA buffer (50 mM Tris-HCl [pH 8.0], 150 mM NaCl, 1% NP-40, 0.5% deoxycholate-Na, 0.1% sodium dodecyl sulphate [SDS]) containing protease inhibitor (Protease Inhibitor Cocktail Set III; CALBIOCHEM). Protein samples were separated by SDS-polyacrylamide gels and electroblotted onto PVDF transfer memebrane (GE Healthcare Bio-sciences). Blots were incubated with a rabbit monoclonal anti-phospho-Akt1(Ser473) antibody (Cell signaling), rabbit monoclonal anti-Akt1 antibody (Cell signaling) or a mouse monoclonal β-actin (ACTB) antibody (Sigma). Protein bands were visualized by enhanced chemiluminescence (ECL) western blotting detection reagents (GE Healthcare Bio-sciences).

### Matrigel Invasion Assay.

NIH3T3 cells transfected either with plasmids expressing PKIB (pcDNA3.1/HA-PKIB) or with mock plasmids were grown to near confluence in DMEM containing 10% FBS. The cells were harvested by trypsinization, washed in DMEM without addition of serum or proteinase inhibitor, and suspended in DMEM at concentration of 5×10⁵/ml. Before preparing the cell suspension, the dried layer of Matrigel matrix (Becton Dickinson Labware) was rehydrated with DMEM for 2 hours at room temperature. DMEM (0.75 ml) containing 10% FBS was added to each lower chambers were processed; cells invading through Matrigel were fixed and stained by Gimsa, as described above.

### Example 2

### Over-Expression of PKIB and NAALADL2 in PC Cells

Among dozens of trans-activated genes that were screened by genome-wide cDNA microarray analysis of HRPC cells (Tamura K et al., Cancer Res 2007 67: 5117-25.), PKIB and NAALADL2 were focused in this invention. PKIB over-expression was confirmed by RT-PCR in five of the nine microdissected HRPC cell populations (FIG. 1A), and NAALADL2 over-expression was confirmed by RT-PCR in five of the nine (FIG. 2A).

The expression of both genes in normal organs including heart, lung, liver, and kidney was minimum, and HRPC cells showed higher expression of the both genes comparing to that of hormone-sensitive or naïve PC cells. Northern-blot analysis using cDNA fragment of PKIB as the probe identified an about 1.3-kb transcript specifically in placenta and PC cell lines, but no expression was observed in any other organs including lung, heart, liver, kidney, and brain (FIG. 1B). Northern-blot analysis using cDNA fragment of NAALADL2 as the probe identified three bands of about 10-kb, 6-kb, and 5-kb transcript specifically in PC cell lines, but no expression was observed in any other organs including lung, heart, liver, kidney, and brain (FIG. 2B).

Database analysis and this RT-PCR spanning the coding region of NAALADL2 suggested that these three bands reflected 3′ UTR variations. Polyclonal antibody specific to human PKIB was generated, and to validate PKIB protein expression in PC cells, immunohitsochemical analysis was performed using 41 clinical PC tissues including 32 hormone-sensitive or naïve PCs and 9 HRPCs. As indicated by northern blot analysis and RT-PCR analysis, PINs (FIG. 1C) and normal prostate epithelium (FIG. 1D, indicated by N) showed week staining (+) for PKIB, and 10/32 (31%) of hormone-naïve PCs showed week or no staining (+) for PKIB as well (FIG. 1D). On the other hand, 22/32 (69%) of hormone-naïve PC (FIG. 1E) and all of six HRPCs (FIG. 1F) showed strong positivity (++ or +++) of PKIB, which was consistent with the result from RT-PCR analysis. Furthermore, all (9/9) of hormone-naïve PC with Gleason Grade 5 (FIG. 1E) showed strong positivity for PKIB as well as HRPCs, and PKIB expression was strongly correlated with Gleason Glade (Table 1, chi-square test, P. 1.35×10⁻⁶), suggesting that PKIB expression could indicate malignant phenotype and poor prognosis of PC.

TABLE 1
Correlation between PKIB expression and Gleason Score (GS) in clinical PC tissues

| | PKIB expression | | | |
|---|---|---|---|---|
| | +++ | ++ | + | − |
| HRPC | 9/9 | — | — | — |
| HNPC | 10/32 | 12/32 | 8/32 | 2/32 |
| Gleason 5 | 7/9 | 2/9 | — | — |
| Gleason 4 | 2/11 | 7/11 | 2/11 | — |
| Gleason 3 | 1/12 | 3/12 | 6/12 | 2/12 |

(+++ vs others : P = 1.3 5 × 10⁻⁶)

### Example 3

### Knockdown of PKIB by siRNA on PC Cell Lines

To investigate a potential growth-promoting role of PKIB aberrant expression, several siRNA-expression vectors were constructed to examine their knockdown effects on a PKIB-expressing PC cell line, 22Rv1, and LNCaP (HP) cells. When si1 and si2 constructs were transfected to 22Rv1 cells (left) and LNCaP (HP) cells (right), a significant knockdown effect was observed by semi-quantitative RT-PCR, but #3si and a negative siRNA construct siEGFP did not (FIG. 3A). After selection in culture medium containing Geneticin, MTT assay (FIG. 3B) and colony formation assay (FIG. 3C) demonstrated that introduction of si1 and si2 in 22Rv1 cells (left) and LNCaP (HP) cells (right) drastically attenuated their cell growth or viability, while that of other siRNAs, which could not affect PKIB expression, did not affected cell growth, indicating that PKIB is likely to play important roles of PC cell viability.

### Example 4

### Knockdown of NAALADL2 by siRNA on PC Cell Lines

To investigate a potential growth-promoting role of NAALADL2 aberrant expression, several siRNA-expression vectors were constructed to examine their knockdown effects on a NAALADL2-expressing PC cell line, 22Rv1 cells. When si#690 construct were transfected to 22Rv1 cells, a significant knockdown effect was observed by semi-quantitative RT-PCR, but other constructs did not (FIG. 4A). After 14-day selection in culture medium containing Geneticin, MTT assay (FIG. 4B) and colony formation assay (FIG. 4C) demonstrated that introduction of si#690 in 22Rv1 cells drastically attenuated their cell growth or viability, while that of other siRNAs, which showed no knockdown effect of NAALADL2 expression, did not affected cell growth, indicating that NAALADL2 is likely to play important roles of cancer cell viability. The synthesized RNA duplex corresponding to si#690 or a negative control siRNA duplex was transfected into another NAALADL2-expressing PC cell line C4-2B cells. RT-PCR showed that si#690 RNA duplex clearly knocked down the endogenous NAALADL2 expression in C4-2B cells (FIG. 4D), and MTT assay demonstrated that si#690 RNA duplex suppressed the growth of C4-2B cells as well (FIG. 4E).

### Example 5

### Subcellular Localization of PKIB and NAALADL2 Proteins

Using the constructed mammalian expression vectors to express tagged-full-length PKIB and NAALADL protein, their subcellular localization were investigated by immunocytochemical analysis using anti-tag antibody. As shown in FIG. 5, exogenous PKIB was localized at the cytoplasm (FIG. 5A), and exogenous NAALADL2 protein was localized at the cytoplasmic membrane (FIG. 5B). NAALADL2 has one transmembrane and it is predicted to localize at the plasma membrane as a type-II membrane protein. This data supported this and NAALADL2 protein is likely to be type-II membrane protein.

### Example 6

### Interaction Between PKIB and PKA-C, and Translocation of PKA-C by Knocking Down PKIB

PKIB belongs to PKI (protein kinase A inhibitor) family and PKIA could inhibit the kinase activity of protein kinase A catalytic subunit (PKA-C) and export PKA-C from the nucleus to the cytoplasm by binding to PKA-C directly through its pusedosubstrate motif (RRNA; SEQ ID NO: 31) (Glass D B et al., J Biol Chem 1986 261: 12166-71, and Wen W et al., J Biol Chem 1994 269: 32214-20.). PKIB also has pusedosubstrate motif (RRNA), but its inhibitory activity is much smaller than that of PKIA (Gamm D M et al., J Biol Chem 1995 270: 7227-32.) and its activity to translocate PKA-C in the cell is unknown. To investigate whether PKIB is involved with PKA-C localization, first the direct interaction between PKIB and PKA-C was validated. PKIB-Myc and HA-PKA-C expression vectors were co-transfected to 22Rv1 cells, and their cell lysates were immunoprecipitated by each of tag antibody. FIG. 5C showed that PKIB-Myc was co-immunoprecipitaed with PKA-C and vice versa, indicating the direct interaction between PKIB and PKA-C.

Next, the subcellular localization of endogenous PKA-C in PC-3 cells or LNCaP(HP) cells which were knocked down for PKIB, was checked by immunocytochemical analysis and cellular fractionation. Immunocytochemical analysis observed most of PKA-C was localized in the cytoplasm and some signal of PKA-C protein in the nucleus (FIG. 5D, left) when control siRNA transfected to PC-3 cells. On the other hand, when siRNA knocked down endogenous PKIB in PC-3 cells, immunocytochemical analysis showed no or very little signal of PKA-C in the nucleus (FIG. 5D, right). Same phenomena were observed in other PKIB-expressing PC cell line LNCaP (HP) cells. To analyze the nuclear PKA-C more quantitatively, the lysates from LNCaP (HP) cells which were treated with PKIB siRNA, were fractionated.

As shown in FIG. 5E, the amount of PKA-C in the nucleus was clearly decreased in PKIB knockdown, comparing with that in control siRNA, while the amount of PKA-C in the cytoplasm was a little increased in PKIB knockdown. These findings implicate that PKIB could facilitate the import of PKA-C to the nucleus or inhibit the export of PKA-C from the nucleus, unlike the nuclear exporting function of PKIA.

### Example 7

### Overexpression of PKIB Promoted PC Cell Growth

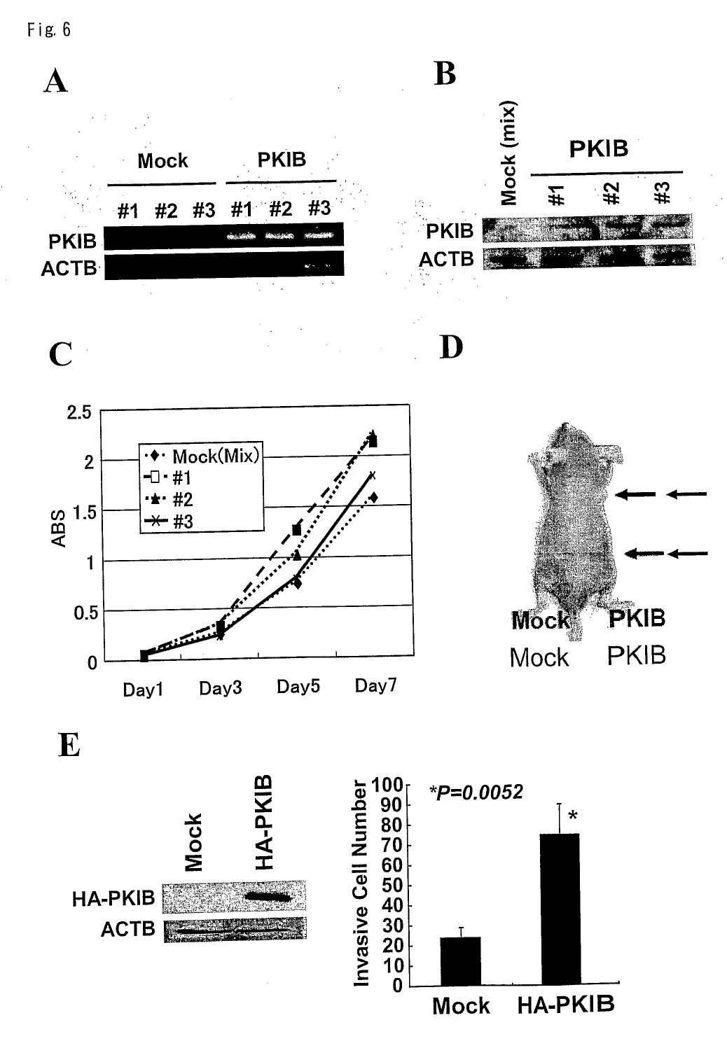

To investigate into the oncogenic function of PKIB, the three clones that constitutively expressed exogenous PKIB were established from DU145 cells, which expressed no or little endogenous PKIB, and those clones were compared their growth with that of Mock DU-145 cells. As shown in, All of three clones constitutively expressed exogenous PKIB (FIG. 6A, FIG. 6B) and they grew more rapidly than Mock cells in vitro (FIG. 6C) and in vivo (FIG. 6D), suggesting the growth-promoting effect of PKIB in prostate cancer. As present immunohistochemical analysis showed, PKIB over-expression was strongly correlated with high Gleason score, which indicated that PKIB could contribute to malignant or invasive potentials of prostate cancer cells. Then, a possible role of PKIB in cellular invasion was examined by Matrigel invasion assay. As shown in FIG. 6E, NIH3T3 cells transfected with PKIB expressing vector significantly enhanced its migration through Matrigel, compared to cells transfected with Mock vector.

### Example 8

### PKIB was Involved with Akt Phosphorylation in PC

Several reports suggested that loss of function of PTEN and activation of Akt are significantly correlated with the progression of prostate cancer and PTEN-PI3K-Akt pathway is likely to play critical roles in HRPC progression and its malignant phenotype (Sellers, W. R. & Sawyers, C. L. (2002) in Somatic Genetics of Prostate Cancer: Oncogenes and Tumor Suppressors ed. Kantoff, P. (Lippincott Williams & Wilkins, Philadelphia), Wang Y, Kreisberg J I, Ghosh P M., Curr Cancer Drug Targets. 2007 September; 7(6):591-604, Lin H K, Yeh S, Kang H Y, Chang C, Proc Natl Acad Sci USA 2001; 98(13):7200-5, Feldman B J, Feldman D, Nat Rev Cancer 2001; 1(1):34-45, Malik S N, Brattain M, Ghosh P M, Troyer D A, Prihoda T, Bedolla R, Kreisberg J I., Clin Cancer Res. 2002; 8(4):1168-71). Then, it was investigated whether PKIB could affect Akt phosphorylation or not. First, knocking down PKIB expression in PC cell lines (LNCaP and PC-3) by RNA dulplex corresponding to si1, the Akt phosphorylation of Ser 473 of Akt was checked by western blot analysis. Knockdown of PKIB by RNA duplex was validated by RT-PCR (data not shown). As a result, PKIB knockdown clearly affected Akt phosphorylation in PC cells (FIG. 7A). Furthermore, as shown in FIG. 7B, it was also observed that over-expression of PKIB in PC cells significantly enhanced Akt phosphorylation at Ser473. It was demonstrated that PKIB directly interacted with PKA-C kinase and could possibly modify its function, and it was confirmed that over-expression of PKA-C kinase also enhanced Akt phosphorylation at Ser473 (FIG. 7A. These findings suggested that PKIB could involve with Akt phosphorylation (Ser473) in PC cells, possibly through the modification of PKA-C kinase function.

Next, as shown in FIG. 7B, it was observed that overexpression of PKIB in PC cells enhanced Akt phosphorylation. It was demonstrated that PKIB could directly interact with PKA-C kinase and modify its function, and it was confirmed that overexpression of PKA-C kinase also enhanced Akt phosphorylation (FIG. 7C). These findings suggested that PKIB could involve with Akt phosphorylation, probably through the modification of PKA-C kinase.

In addition, to confirm whether Ser473 phosphorylation of Akt by PKA-C and/or PKIB directly, in vitro kinase assay was performed by using recombinant PKIB, PKA-C kinase and Akt proteins. As shown in FIG. 7D, phosphorylated Ser473-specific antibody detected phosphorylated Akt when PKA-C kinase reacted with recombinant Akt, and the phosphorylation level of Akt (Ser473) was clearly enhanced by addition of PKIB. This suggested that PKA-C kinase with PKIB directly and effectively could phosphorylate Akt Ser473, which could contribute to the growth promotion and progression of PCs cells.

### Example 9

### Akt Phosphorylation and PKIB Over-Expression in PC Tissues

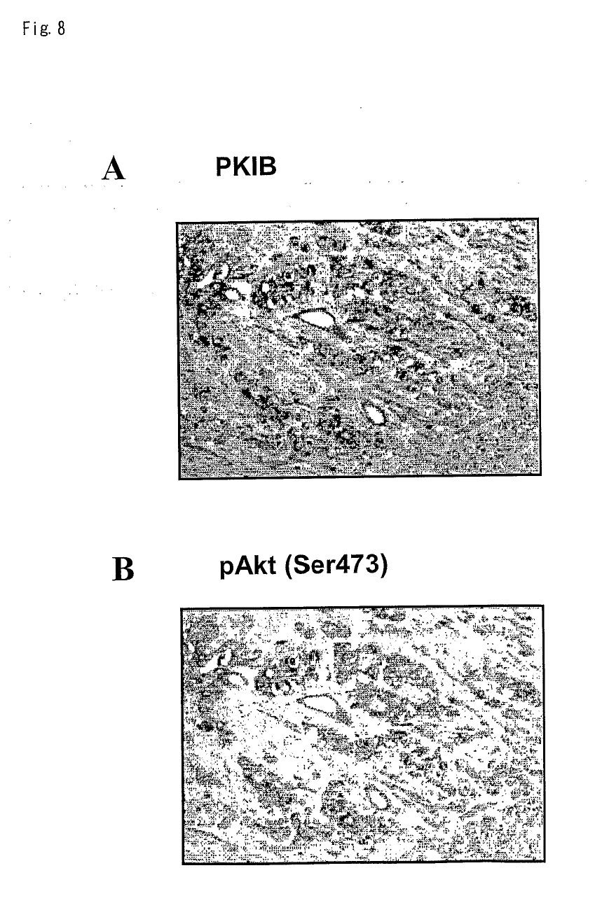

Finally, the correlation between PKIB expression and Akt phosphorylation at Ser473 was examined in clinical PC tissues. FIG. 8 showed the representative pictures of PC tissues, comparing the staining pattern of PKIB and that of pAkt (Ser473) in the face-on-face slides of PC tissues, indicating the correlation between PKIB expression and Akt phosphorylation in this PC tissue. Table 2 summarized the correlation between PKIB expression and Akt phosphorylation (P=0.0156, chi²-test) in clinical PC tissues.

TABLE 2
The correlation between PKIB expression and Akt
phosphorylation at Ser473 in clinical PC tissues

| | pAkt (Ser473) | | |
|---|---|---|---|
| PKIB expression | ++ | + | − |
| ++ (n = 22) | 8 (36%) | 10 (45%) | 4 (18%) |
| + (n = 20) | 2 (10%) | 11 (55%) | 7 (35%) |
| − (n = 8) | 0 | 2 (25%) | 6 (75%) |

(P = 0.01 56. chi²-test)

### Discussion

In this invention, two molecular targets for prostate cancer, especially for hormone-refractory prostate cancer, were identified using genome-wide gene expression profiles of clinical HRPC cells. Prostate cancer shows relatively good prognosis, and hormone depletion therapy is effective in most of relapsed or advanced PC. But once HRPC cells emerge, very few options for PC patient care are available. As a result, the prior art has recognized a need for the identification of novel molecular targets for HRPC patients and development of novel therapies for HRPCs by targeting these novel molecules.

PKIB belongs to PKI (protein kinase A inhibitor) family, and PKIA could inhibit the kinase activity of protein kinase A catalytic subunit (PKA-C) and export PKA-C from the nucleus to the cytoplasm by binding to PKA-C directly (Glass D B et al., J Biol Chem 1986 261: 12166-71, and Wen W et al., J Biol Chem 1994 269: 32214-20.). Protein kinase A (PKA), cAMP-dependent protein kinase A, is often considered essential for mediating the wide range of physiological or pathological effects initiated by cAMP, and coupling with G protein, a number of ligand and receptor systems activate PKA signaling pathway, and its activation is associated with the control of cell growth and differentiation (Tasken K et al., Physol Review 2004 84: 137-67, and Stork P J et al., Trends Cell Biol 2002 12:258-66.).

In prostate cancer, several reports suggested its involvement with androgen-independent growth and neuroendocrine differentiation (Cox M E et al., J Biol Chem 2000 275: 13812-8.), and cross-talk between PKA pathway and AR pathway is suggested to be involved with androgen-independent growth of HRPC cells (Stork P J et al., Trends Cell Biol 2002 12:258-66, and Sadar M D, J Biol Chem 1999 274: 7777-83.).

In the present invention, it was demonstrated that PKIB could facilitate the nuclear localization of PKA-C and promote PC cell growth, rather that inhibit PKA-C activity or PKA pathway as a PKI family member. Previous report suggested PKIB has some inhibitory activity of PKA-C in vitro, but its Km is much higher than PKIA, and facilitating nuclear import of PKA-C or inhibiting its nuclear export may be dominant as the function of PKIB in PC cells. Furthermore, PKIB could strongly associate Akt Ser473 phosphorylation which could play critical roles in the aggressive and malignant phenotype of CRPCs.

NAALADL2 is a novel type II membrane protein and belongs to glutamate carboxypeptidase II (GCPII) family. The prostate form of GCPII, termed prostate-specific membrane antigen (PSMA) is expressed in prostate cancer and increased levels of PSMA are associated with PC progression and HRPC (Rajasekaran A K et al., Am J Physiol Cell Physiol 2005 288: C975-81, and Murphy G P et al., Prostate 2000 42: 145-9.). Considering its homology with PSMA and its similar expression pattern, this novel molecule NAALADL2 should be termed as “PSMA2”. PSMA is the target of a FDA-approved prostate cancer-imaging agent, the 111In-labeled 7E11 monoclonal antibody (Prostascint, Cytogen, Princeton, N.J.), and PSMA is targeted by monoclonal antibodies such as J591, which is in clinical trials for specific delivery of imaging agent or therapeutics to PSMA-expressing cells (Murphy G P et al., Prostate 2000 42: 145-9, and Holmes E H, Expert Opin Investig Drugs 2001 10: 511-9.).

In addition to its character as a tumor marker, PSMA has GPC activity whose substrates include poly-gamma-glutamated folates (Zhou J et al., Nature Review Drug Disc 2005 4: 1015-26.). The enzymatic activity of PSMA can be exploited for the design of prodrugs, in which an inactive glutamated form of the drug is selectively cleaved and thereby activated only at cells expressing PSMA (Denny W A et al., Eur J Med Chem 2001 36: 577-95.) However, how PSMA is associated with prostate cancer progression is completely unknown, and possibility of targeting PSMA function or activity itself is yet to be known. NAALADL2 is highly expressed in HRPC cells and its expression in normal adult organs is very limited as shown in FIG. 2B. In addition to its restrictive expression as a tumor marker, it is likely to be involved with PC viability or growth, which is supported by this siRNA experiment. Hence, specific monoclonal antibody to PSMA2 would be applicable to PC therapy, as well as a tumor marker, by blocking PSMA2 activity.

### INDUSTRIAL APPLICABILITY

The expression of human genes PKIB and NAALADL2 are markedly elevated in prostate cancer, especially hormone-refractory prostate cancer or castration-resistant prostate cancer, as compared to normal organs. Accordingly, these genes can be conveniently used as diagnostic markers of prostate cancer and the proteins encoded thereby may be used in diagnostic assays of prostate cancer.

The present inventors have shown that the cell growth is suppressed by double-stranded molecules that specifically target the PKIB and NAALADL2 gene. Thus, these novel double-stranded molecules are useful for the development of anti-cancer pharmaceuticals. For example, agents that block the expression of PKIB or NAALADL2 protein or prevent their activity have therapeutic utility as anti-cancer agents, particularly anti-cancer agents for the treatment of prostate cancer, especially hormone-refractory prostate cancer or castration-resistant prostate cancer.

Furthermore, either the PKIB or NAALADL2 polypeptide is a useful target for the development of anti-cancer pharmaceuticals. For example, agents that bind PKIB or NAALADL2, or block the expression of PKIB or NAALADL2, or prevent its activity, or inhibit the binding between PKIB and PKA-C, or anti-NAALADL2 antibodies may find therapeutic utility as anti-cancer agents, particularly anti-cancer agents for the treatment of prostate cancer, especially hormone-refractory prostate cancer or castration-resistant prostate cancer.

While the invention has been described in detail and with reference to specific embodiments thereof, it will be apparent to one skilled in the art that various changes and modifications can be made therein without departing from the spirit and scope of the invention.

1. An isolated double-stranded molecule, which when introduced into a cell, inhibits in vivo expression of PKIB or NAALADL2 and cell proliferation, which double stranded molecule comprises a sense strand and an antisense strand complementary thereto, hybridized to each other to form the double-stranded molecule.

2. The double-stranded molecule of claim 1, wherein the sense strand comprises the sequence corresponding to a target sequence selected from the group consisting of SEQ ID NOs: 16, 17 and 19.

3. The double-stranded molecule of claim 2, which has a length of between about 19 and about 25 nucleotides.

4. The double-stranded molecule of claim 2, which consists of a single polynucleotide comprising both the sense and antisense strands linked by an intervening single-strand.

5. The double-stranded molecule of claim 4, which has the general formula 5′-[A]-[B]-[A′]-3′, wherein [A] is the sense strand comprising a sequence selected from the group consisting of SEQ ID NOs: 16, 17 and 19, [B] is the intervening single-strand consisting of 3 to 23 nucleotides, and [A′] is the antisense strand comprising a sequence complementary to [A].

6. A vector expressing the double-stranded molecule of claim 1.

7. The vector of claim 6, wherein the double-stranded molecule has the general formula 5′-[A]-[B]-[A′]-3′, wherein [A] is the sense strand comprising a sequence selected from the group consisting of SEQ ID NO: 16, 17 and 19, [B] is an intervening single-strand consisting of 3 to 23 nucleotides, and [A′] is the antisense strand comprising a sequence complementary to [A].

8. A method for treating cancer comprising the step of administering at least one isolated double-stranded molecule inhibiting the expression of a PKIB gene or a NAALADL2 gene in a cell over-expressing the gene, which double stranded molecule comprises a sense strand and an antisense strand complementary thereto, hybridized to each other to form the double-stranded molecule.

9. The method of claim 8, wherein the sense strand comprises a sequence corresponding to a target sequence selected from the group consisting of SEQ ID NOs: 16, 17 and 19.

10. The method of claim 9, wherein the double-stranded molecule has a length of between about 19 and about 25 nucleotides in length.

11. The method of claim 8, wherein the double-stranded molecule consists of a single polynucleotide comprising both the sense strand and the antisense strand linked by an interventing single-strand.

12. The method of claim 11, wherein the double-stranded molecule has the general formula 5′-[A]-[B]-[A′]-3′, wherein [A] is the sense strand comprising a sequence selected from the group consisting of SEQ ID NOs: 16, 17 and 19, [B] is an intervening single strand consisting of 3 to 23 nucleotides, and [A′] is the antisense strand comprising a sequence complementary to [A].

13. The method of claim 8, wherein the double-stranded molecule is encoded by a vector.

14. The method of claim 13, wherein the double-stranded molecule encoded by the vector has the general formula 5′-[A]-[B]-[A′]-3′, wherein [A] is the sense strand comprising a sequence selected from the group consisting of SEQ ID NOs: 16, 17 and 19, [B] is an intervening single-strand consisting of 3 to 23 nucleotides, and [A′] is the antisense strand comprising a sequence complementary to [A].

15. The method of claim 8, wherein the cancer to be treated is prostate cancer or hormone-refractory prostate cancer or castration-resistant prostate cancer.

16. A composition for treating cancer, comprising at least one isolated double-stranded molecule which inhibits the expression of PKIB or NAALADL2, which double stranded molecule comprises a sense strand and an antisense strand complementary thereto, hybridized to each other to form the double-stranded molecule.

17. The composition of claim 16, wherein the sense strand comprises a sequence corresponding to a target sequence selected from the group consisting of SEQ ID NOs: 16, 17 and 19.

18. The composition of claim 17, wherein the double-stranded molecule has a length of between about 19 and about 25 nucleotides.

19. The composition of claim 16, wherein the double-stranded molecule consists of a single polynucleotide comprising the sense strand and the antisense strand linked by an intervening single-strand.

20. The composition of claim 19, wherein the double-stranded molecule has the general formula 5′-[A]-[B]-[A′]-3′, wherein [A] is the sense strand sequence comprising a sequence selected from the group consisting of SEQ ID NOs: 16, 17 and 19, [B] is an intervening single-strand consisting of 3 to 23 nucleotides, and [A′] is the antisense strand comprising a sequence complementary to [A].

21. The composition of claim 16, wherein the double-stranded molecule is encoded by a vector and contained in the composition.

22. The composition of claim 21, wherein the double-stranded molecule has the general formula 5′-[A]-[B]-[A′]-3′, wherein [A] is the sense strand comprising a sequence selected from the group consisting of SEQ ID NOs: 16, 17 and 19, [B] is an intervening single-strand consisting of 3 to 23 nucleotides, and [A′] is the antisense strand comprising a sequence complementary to [A].

23. The composition of claim 16, wherein the cancer to be treated is the prostate cancer or hormone-refractory prostate cancer or castration-resistant prostate cancer.

24. A method for diagnosing prostate cancer, said method comprising the steps of:
(a) determining the expression level of the gene in a subject-derived biological sample by any one of the method selected from the group consisting of
(i) detecting the mRNA comprising a sequence corresponding to SEQ ID NO: 1, 3 or 5,
(ii) detecting the protein comprising the amino acid sequence of SEQ ID NO: 2 or 4, and
(iii) detecting the biological activity of the protein comprising the amino acid sequence of SEQ ID NO: 2 or 4; and
(b) relating an increase of the expression level compared to a normal control level of the gene to the prostate cancer.

25. The method of claim 24, wherein the prostate cancer is hormone-refractory prostate cancer or castration-resistant prostate cancer.

26. The method of claim 24, wherein the expression level is at least 10% greater than the normal control level.

27. The method of claim 24, wherein the expression level is determined by detecting hybridization of a probe to a gene transcript of said subject-derived biological sample.

28. The method of claim 27, wherein the hybridization step is carried out on a DNA array.

29. The method of claim 24, wherein the expression level is determined by detecting the binding of an antibody against the protein comprising the amino acid sequence of SEQ ID NO: 2 or 4.

30. The method of claim 29, wherein the antibody binds to a polypeptide consisting of SEQ ID NO: 32, 33 or 34.

31. The method of claim 24, wherein the subject-derived biological sample comprises biopsy, sputum, blood or urine.

32. An antibody which binds to a protein comprising an amino acid sequence of SEQ ID NO: 33 or 34.

33. A composition for detecting prostate cancer, which comprises an antibody which binds to a protein comprising an amino acid sequence of SEQ ID NO:2 or 4.

34. The composition of claim 33, wherein the antibody binds to SEQ ID NO: 32, 33 or 34.

35. A method of screening for a compound for treating or preventing prostate cancer, said method comprising the steps of
a) contacting a test compound with a polypeptide encoded by a polynucleotide of PKIB or NAALADL2;
b) detecting the binding activity between the polypeptide and the test compound; and
c) selecting the test compound that binds to the polypeptide.

36. A method of screening for a compound for treating or preventing prostate cancer, said method comprising the steps of:
a) contacting a test compound with a polypeptide encoded by a polynucleotide of PKIB or NAALADL2;
b) detecting the biological activity of the polypeptide of step (a); and
c) selecting the test compound that suppresses the biological activity of the polypeptide encoded by the polynucleotide of PKIB or NAALADL2 as compared to the biological activity of said polypeptide detected in the absence of the test compound.

37. A method of claim 36, wherein the biological activity is the facilitation of the cell proliferation or PKA-C nuclear accumulation activity.

38. A method of screening for a compound for treating or preventing prostate cancer, said method comprising the steps of:
a) contacting a candidate compound with a cell expressing PKIB or NAALADL2; and
b) selecting the candidate compound that reduces the expression level of PKIB or NAALADL2 in comparison with the expression level detected in the absence of the test candidate compound.

39. A method of screening for a compound for treating or preventing prostate cancer, said method comprising the steps of
a) contacting a candidate compound with a cell into which a vector, comprising the transcriptional regulatory region of PKIB or NAALADL2 and a reporter gene that is expressed under the control of the transcriptional regulatory region, has been introduced;
b) measuring the expression or activity of said reporter gene; and
c) selecting a candidate compound that reduces the expression or activity level of said reporter gene as compared to a control.

40. A method of screening for a compound for treating or preventing prostate cancer, said method comprising the steps of:
a) contacting a PKIB polypeptide or functional equivalent thereof with PKA-C polypeptide or functional equivalent thereof in the presence of a test compound;
b) detecting the binding between the polypeptides; and
c) selecting the test compound that inhibits the binding between the polypeptides.

41. The method of claim 40 wherein the functional equivalent of PKIB polypeptide comprises the polypeptide consisting of SEQ ID NO: 31.

42. The method of claim 40, wherein the functional equivalent of PKA-C polypeptide comprises amino acid sequence of PKIB binding domain.

43. A method of screening for a compound for treating or preventing prostate cancer, said method comprising the steps of:
(a) incubating a PKIB polypeptide or a functional equivalent thereof, a PKA-C polypeptide or a functional equivalent thereof and Akt in the presence of a test compound under conditions suitable for the phosphorylation of Akt by the PKIB polypeptide
(b) detecting a phosphorylation level of the Akt,
(c) comparing the phosphorylation level of the Akt measured in step (b) to a control level, and
(d) selecting a compound that decreases the phosphorylation level the Akt as compared to the control level.

44. A method of claim 43, wherein the phosphorylation level of Akt is detected at the 473 serine residue of amino acid sequence of SEQ ID NO: 35.

45. The method of claim 43, wherein the PKIB polypeptide or the functional equivalent thereof, the PKA-C polypeptide or the functional equivalent thereof and Akt are expressed in a cell, and incubated in the presence of a test compound by contacting the cell or lysate thereof with a test compound.

46. The method of any one of the claims 35, 36, 38, 39, 40 and 43, wherein the prostate cancer is hormone-refractory prostate cancer or castration-resistant prostate cancer.


SEQUENCE LISTING (36 sequences)

SEQ ID NO: 1 - DNA, 1909 bp, Homo sapiens
ggaaaaaagctaccgtggcacgtgccccggggagtgatgcggtggcccggtttgcacggagagcgggacaccgcctggggagccgcgctcgtagcctgggggcgggacgcggcggccgctccctccgcccccgagtagctgccaccgcctggctgcgccccagcccagtagctcagacgcggccgcatcccggtggactgtagaggcggcagcgagctagagcccgagtcgcagctccgggccgcagagcgctgggcgagcgcgagcgccagggcaccggcagggcaggcagctgcgcgcggctggagtcatgctatactgaaaagacacttcatcaagataactctgggagaagcagaaaaccctgtgccagggacaggaaagataggagaaagaaagtttatcagaattttttaaacctgtctcagaaataacaacatattttaatcagagatttatgttgctatgaggacagattcatcaaaaatgactgacgtggagtctggggtcgccaattttgcatcttcagcaagggcaggccgccggaatgccttaccagacatccagagttcagctgccacagacggaacctcagatttgcccctcaaactggaggctctctccgtgaaggaagatgcaaaagagaaagatgaaaaaacaacacaagaccaattggaaaagcctcaaaatgaagaaaaatgaaggctcataatctatcaagagtgctgaatttctgcatgttgaaagacttagtggttctgttttcttgagacatttaatctggtggtaactgtggtaacattgcagccctaagcagcatgtgtatattagataattgtgttgtgatgctactcactttgattgcaatgatgatgtccaaggtaagctattaaaaggcaggttacttccaaatcgcactgaaggaaaaggttaagaataatacatgatcacagaaatgcataccactgtctgtaaacccaacaaaattcactgttctcttttggatttatttagcctgatgtatttttaattcaatttttatggtgatgggcaaatcattcttggtaaatgtaaatcaaacatgattgatttaaaacttcatggaatttgtagaaaattatggacatttttggtgagaaagaacaatagtcaaaactcacatggatagagtgtgtttgttttttgccaaaaatgccccagactttttcccaaacctcaaaaacgtcttggaaaaattgtaaaagtttgataacagaaacatctttaggatatttttgtctgacatattttgcttctagtatgtgcctactgtgatttttttcatgtggaaaatgcaaaatttgtaacaaaatggttatatggaacatgcctattaaatgaattttactatcttccctaactttggtctgtgtatgtgtgtgtgttttactttaatatgaattatacaaaatactagttgttttacactctcttttcttattcttagggcttttgtgtatgtctgacttgtttttaaataacttcctcagcaatgcagaccttaatttttatatttttttaaagtagctaacatagcagtaggcacttaagcatttagtcaatgatattggtagaaatagtaaaatacatcctttaaatatatatctaagcatatattttaaaaggagcaaaaataaaaccaaagtgttagtaaattttgatttattagatattttagaaaaataatagaattctgaagttttaaaaatgtcagtaattaatttattttcattttcagaaatatatgcatgcagttatgttttatttgattgttgacttaggctatgtctgtatacagtaaccaaataaactctttcactattaaagagatttcttactgaaaaaaaaaaaaaaaaaaaaaaaaaaaaaa

SEQ ID NO: 2 - protein, 78 aa, Homo sapiens
MRTDSSKMTDVESGVANFASSARAGRRNALPDIQSSAATDGTSDLPLKLEALSVKEDAKEKDEKTTQDQLEKPQNEEK

SEQ ID NO: 3 - DNA, 4912 bp, Homo sapiens
acagtagaaagtcagaaggtcacaaagcttgcagggtaagtgacacaacttgaaactgcttggccctctttaaaaagaaataataaaatgggagagaatgaagcaagtttacctaacacgtctttgcaaggtaaaaagatggcctatcagaaggtccatgcagatcaaagagctccaggacactcacagtacttagacaatgatgaccttcaagccactgcccttgacttagagtgggacatggagaaggaactagaggagtctggttttgaccaattccagctagacggtgctgagaatcagaacctagggcattcagagactatagacctcaatcttgattccattcaaccagcaacttcacccaaaggaaggttccagagacttcaagaagaatctgactacattacccattatacacgatctgcaccaaagagcaatcgctgcaacttttgccacgtcttaaaaatactttgcacagccaccattttatttatttttgggattttgataggttattatgtacatacaaattgcccttcagatgctccatcttcaggaacagttgatcctcagttatatcaagagattctcaagacaatccaggcagaagatattaagaagtctttcagaaatttggtacaactatataaaaatgaagatgacatggaaatttcaaagaagattaagactcagtggacctctttgggcctagaagatgtacagtttgtaaattactctgtgctgcttgatctgccaggcccttctcccagcactgtgactctgagcagcagtggtcaatgctttcatcctaatggccagccttgcagtgaagaagccagaaaagatagcagccaagacctgctctattcatatgcagcctattctgccaaaggaactctcaaggctgaagtcatcgatgtgagttatggaatggcagatgatttaaaaaggattaggaaaataaaaaacgtaacaaatcagatcgcactcctgaaattaggaaaattgccactgctttataagctttcctcattggaaaaggctggatttggaggtgttcttctgtatatcgatccttgtgatttgccaaagactgtgaatcctagccatgataccttcatggtgtcactgaatccaggaggagacccttctacgcctggttacccaagtgtcgatgaaagttttagacaaagccgatcaaacctcacctctctattagtgcagcccatctctgcacccctcgttgcaaaactgatctcttcgccaaaagctagaaccaaaaatgaagcgtgtagctctctagagcttccaaataatgaaataagagtcgtcagcatgcaagttcagacagtcacaaaattgaaaacagttactaatgttgttggatttgtaatgggcttgacatctccagaccggtatatcatagttggcagccatcatcacactgcacacagttataatggacaagaatgggccagtagtactgcaataatcacagcgtttatccgtgccttgatgtcaaaagttaagagagggtggagaccagaccgaactattgttttctgttcttggggaggaacagcttttggcaatattggctcatatgaatggggagaggatttcaagaaggttcttcagaaaaatgttgtggcttatattagcctccacagtcccataagggggaactctagtctgtatcctgtagcatcaccatctcttcagcaactggtagtagagaaaaataatttcaactgtaccagaagagcccagtgcccagaaaccaatatcagttctatacagatacaaggtgatgctgattatttcatcaaccatcttggagttcccatcgtgcagtttgcttacgaggacatcaaaacattagagggtccaagttttctctccgaggcccgtttttctacacgagcaacaaaaattgaagaaatggatccctctttcaaccttcatgaaaccattactaagctctcaggagaagtgattttgcaaattgccaacgaacctgttctgccctttaatgcacttgatatagctttagaagttcaaaacaaccttaaaggtgatcaacccaacactcatcaactgttagccatggcgttacgcctgcgggagagtgctgaactttttcagtctgatgagatgcgacctgctaatgatcccaaggagagagcacccatccgcatccggatgctgaatgacattctccaagacatggagaaaagctttctggtaaagcaggcaccaccaggtttttatagaaacatcctctaccaccttgatgaaaagacaagccggttttcaatacttatagaggcttgggaacactgcaaaccccttgcatcaaatgagacccttcaagaagccctgtcagaggtgttgaacagcattaattcagctcaggtttacttcaaagcaggacttgatgtgttcaagagtgtcttggatgggaagaattgagaaaactctgagcatttttaaaagtttgtttacaattccacaagcaaaagctctaatttaaccagattttctgacattgaaggcttattttccccaatggctttttgacaagtataaagctattattacattgtattttttaaatgtaaatatagaaagaacattttgcacatttaatatttttcttatatctacatttctgaatatgtaaagcaagttattgaaataggacttaagaattacctattaaaaagaaatctgatatataaaaatatgtactattttaaggaaaaatttccaagaagttctggactatctttgttcctatggggagggcatataggaacacagtttattctctctgatagagctattaatgacttagtttctgtaaaagaaatggagagttgatatggttcagattaacttcactattaagtgttcaatatgaagaattcaagtattctgactgagtgattggttgacctaaaccactttgaatgtttctattttatgaaatgaagtttctcttcttaacacaactagatgtagtaatacactggttatgaaattgtatttttttaagtattaatgaaaaaagagccataagcattccaggagaaaatctcaagggagctacatagagcaatttaaatgcaaatttttttcctaacaacttacaaggtgactagctttgaaacccctaatttgcctcagttgattttctaagaatttcaggagtgatgtatgtcttaagagggagaaaaaaatatttcttattactttttctcttgtttctgttggaaacactgaagcagggactctaaaatgaaagcatctcacattggttttctttcttgtatcttttctgaaactccttgctgtaaggcagctttctcagagtactatatattttcaacagtaaagtagcagagtttctctttgaaacccaaaatgtcttctaaagaatcaaatttctatttctgcctctgacaaaaagaacttactatgaaagaaatagttgttttatcaataaaagccccttaatattatgaagaaacttttcatattttttcttctttatccttaattgtgactaagatggaaatctagaaaatattcatgcttctaagtttctgcagtgttactgtaagagagtgtctcgtctacacttcagttcttctctcatgtggaaaactaagctttttatattgacattgccattgaatagctctagcaactattattattcccccaaaccctaaaattctacctattaggtgtcaggttaaagttctaatttatctttagaaatttatgtgaaaaatggttttcctatactggataaagcaattccccttaggaaacatagcctcagagtatttcatataaattttctctatctaattcaaacatatctccccatgatacaccgccagagtgacattgcagacagtctgtgtatgtttaaattttcactttttatggatgagcataaacctagtcttagtttaaaggcaaactaaagttgaggggaaataattaatcaatactgattaattgatttataaggttcaacactgtctaccctaaataatttttatatgctcaaggcaaagcagggaatagaggcaattagctagataattcaagcagaaatccattttcatcgaaattgcctgtgtgcacatatttttagatgaaaaatgaaactaccttacaatattatttttttaaccaatgtatttgtttgcaaacatttgccatgttgaagagtgtgtataggaaaggtctagaaataaataggcttgtgcatacctaggcattcatgctctttcttcctcttcagagatcatcagaaaagaggtgggaatacacaagcaggtgcatgtaagcccaaaagctagaaagccttatatttaaccaaaggttgatgtgttcattccagttattcacttacagatgaaactaaaacaaataaaggcaagctattatcagtttggtagttcattattttagaacaaatttctaaattaatttattcccataacctacaatagcactgagaacagaaaacatgtttcttcttcattttgtttaaagattgcgtacctaggtaagtcacactgtatagataaaaaccttcttctgtattcctcacctctaaattatatgttatttgctatttgaaaactgtagttaactatggttttttattagtctattaaaagatacgtggagatattaaattatacctaaagcagacgtccaacaaattctgtacatgctgctttacatctttgcaacaaacctcttaactagcagcattatttctatagtgtgattacctgagggtgcctccaaatgtatatcatttcactcccctcccctccccacccccaatagattcaaataaataaaatcctgagcaaattaa

SEQ ID NO: 4 - protein, 795 aa, Homo sapiens
MGENEASLPNTSLQGKKMAYQKVHADQRAPGHSQYLDNDDLQATALDLEWDMEKELEESGFDQFQLDGAENQNLGHSETIDLNLDSIQPATSPKGRFQRLQEESDYITHYTRSAPKSNRCNFCHVLKILCTATILFIFGILIGYYVHTNCPSDAPSSGTVDPQLYQEILKTIQAEDIKKSFRNLVQLYKNEDDMEISKKIKTQWTSLGLEDVQFVNYSVLLDLPGPSPSTVTLSSSGQCFHPNGQPCSEEARKDSSQDLLYSYAAYSAKGTLKAEVIDVSYGMADDLKRIRKIKNVTNQIALLKLGKLPLLYKLSSLEKAGFGGVLLYIDPCDLPKTVNPSHDTFMVSLNPGGDPSTPGYPSVDESFRQSRSNLTSLLVQPISAPLVAKLISSPKARTKNEACSSLELPNNEIRVVSMQVQTVTKLKTVTNVVGFVMGLTSPDRYIIVGSHHHTAHSYNGQEWASSTAIITAFIRALMSKVKRGWRPDRTIVFCSWGGTAFGNIGSYEWGEDFKKVLQKNVVAYISLHSPIRGNSSLYPVASPSLQQLVVEKNNFNCTRRAQCPETNISSIQIQGDADYFINHLGVPIVQFAYEDIKTLEGPSFLSEARFSTRATKIEEMDPSFNLHETITKLSGEVILQIANEPVLPFNALDIALEVQNNLKGDQPNTHQLLAMALRLRESAELFQSDEMRPANDPKERAPIRIRMLNDILQDMEKSFLVKQAPPGFYRNILYHLDEKTSRFSILIEAWEHCKPLASNETLQEALSEVLNSINSAQVYFKAGLDVFKSVLDGKN

SEQ ID NO: 5 - DNA, 1724 bp, Homo sapiens
ttaaaaaagcctcgttagaatttgctattcgaaaagaccttaaaaaccctcacagagttctaaacatcccattcattgaaaatacttttcagttaagtagatttgttttgtgcacttcacaacttttaggtgacatgaatttgaagcgtagcaaaagaaatgtataaagatagccttttctggtcattaccatgtctactcaagtttctgttttctaggtacactctagcattgtaactttttccccctgagaagtaattttaagatctatcagtctcaatttaaatgatctgttaatcagccagagttttagtttcataatatcgttccattgcctgacaaagatatacacactgaagtgcctttagcagacctgggaccgtcaagaatcttgttaccctgattattgcaagatgacatatttcttaagccatttataatctcatattcgggttgaatctgtatttacaaataaaagggttaaattgaggcagtttcaagcagcatttaggaaaatgaagtggcttcaaattttagtgtttctggttacattattttgtttgaattatacaattacataattttctgtaaccaaaatggtaattttgatggattttttaaatgccaaaatccaatcatcaaggccaaagaaatgcatgattactctgatttcttatgcaccattcagtcaagacttaactcagaggcagttgattcagtgcttacatctagacaaagctttaatgagtgcagacccagcctaacagtatttcatctaatttctttgatggcttaagccaataagcactgaggtagcttttctcagaaggaaacagattttattttccaggggctaattaatatgcaccacctaagtccccaaaggatcacacaggtgcctgtatcatgccatatgtcatgtgctatattcctgcaagctcaagagattaatatacaatcattattatgaattattatgttgcattgaagttaatgtcggtcttttgttctaattaaagtacaaacgtggcatctgaatagaagcttagctagagaagtggagttagagtccctattttaatgaactacatatatttttcaatcaaaatgtgtaattatttaattatctagcctgctcagtaatcataactctccaacttcttcaaagggcctttattcaatatgttaccactcatatggtcattacttggtaagtgttttatattttgaatttcttagatgctttcagtgtatgtgctagtgttttattaatattaaaaatttttgtttacttatatatcaagctgctgcaaatggactaccttattttaaaagttagaataaaatgctattactctctaaacagaactttagcatatctgtttgataagaaatacacaaacaaaatatttttctatgctattgcaaattgtccgcaggaagcaaaatgccagatggttaagtgtagctcacataattatattccaaaggtgttataaacaattctatttagcatctgagataggtctatattaggcaatttcatgtttacatttctaacagaaaggtttaatggcaaatattccttatattctaacttgctacttggagaatatggatattctgaaaagaaaaaccctttctagaacactgtgcaggactaatttttttttaatagacatccagaaaatagcctgggcaacaaagtgagaccctgtctctct

SEQ ID NO: 6 - DNA, 20 bp, Artificial
  An artificially synthesized primer for PCR
ttggcttgactcaggattta

SEQ ID NO: 7 - DNA, 20 bp, Artificial
  An artificially synthesized primer for PCR
atgctatcacctcccctgtg

SEQ ID NO: 8 - DNA, 24 bp, Artificial
  An artificially synthesized primer for PCR
ggcacatactagaagcaaaatacg

SEQ ID NO: 9 - DNA, 22 bp, Artificial
  An artificially synthesized primer for PCR
gatgggcaaatcattcttggta

SEQ ID NO: 10 - DNA, 23 bp, Artificial
  An artificially synthesized primer for PCR
gaaagcatctcacattggttttc

SEQ ID NO: 11 - DNA, 23 bp, Artificial
  An artificially synthesized primer for PCR
gggtttcaaagagaaactctgct

SEQ ID NO: 12 - DNA, 20 bp, Artificial
  An artificially synthesized primer for PCR
gaagcaaaatgccagatggt

SEQ ID NO: 13 - DNA, 23 bp, Artificial
  An artificially synthesized primer for PCR
tcctgcacagtgttctagaaagg

SEQ ID NO: 14 - DNA, 20 bp, Artificial
  An artificially synthesized primer for northern blot probe
ccagtgcccagaaaccaata

SEQ ID NO: 15 - DNA, 22 bp, Artificial
  An artificially synthesized primer for northern blot probe
tcaattcttcccatccaagaca

SEQ ID NO: 16 - DNA, 19 bp, Artificial
  An artificially synthesized target sequence for siRNA
gccctaagcagcatgtgta

SEQ ID NO: 17 - DNA, 19 bp, Artificial
  An artificially synthesized target sequence for siRNA
gcagtaggcacttaagcat

SEQ ID NO: 18 - DNA, 19 bp, Artificial
  An artificially synthesized target sequence for siRNA
gatgcaaaagagaaagatg

SEQ ID NO: 19 - DNA, 19 bp, Artificial
  An artificially synthesized target sequence for siRNA
gactcagtggacctctttg

SEQ ID NO: 20 - DNA, 19 bp, Artificial
  An artificially synthesized target sequence for siRNA
gtcatcgatgtgagttatg

SEQ ID NO: 21 - DNA, 19 bp, Artificial
  An artificially synthesized target sequence for siRNA
gagtcgtcagcatgcaagt

SEQ ID NO: 22 - DNA, 19 bp, Artificial
  An artificially synthesized target sequence for siRNA
gaagcagcacgacttcttc

SEQ ID NO: 23 - DNA, 19 bp, Artificial
  An artificially synthesized target sequence for siRNA
gacucaguggaccucuuug

SEQ ID NO: 24 - DNA, 21 bp, Artificial
  An artificially synthesized target sequence for siRNA
caaagagguccacugagucuu

SEQ ID NO: 25 - DNA, 22 bp, Artificial
  An artificially synthesized target sequence for siRNA
gcagcacgacuuctuucaagtt

SEQ ID NO: 26 - DNA, 21 bp, Artificial
  An artificially synthesized target sequence for siRNA
cuugaagaagucgugcugctt

SEQ ID NO: 27 - DNA, 21 bp, Artificial
  An artificially synthesized target sequence for siRNA
gcccuaagcagcauguguaua

SEQ ID NO: 28 - DNA, 21 bp, Artificial
  An artificially synthesized target sequence for siRNA
uacacaugcugcuuagggcuu

SEQ ID NO: 29 - DNA, 2689 bp, Homo sapiens
gatcttgggctgaggttcccgggcgggcgggcgcggagagacgcgggaagcaggggctgggcgggggtcgcggcgccgcagctagcgcagccagcccgagggccgccgccgccgccgcccagcgcgctccggggccgccggccgcagccagcacccgccgcgccgcagctccgggaccggccccggccgccgccgccgcgatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaggggcatgcctgtgcccccatgggttttcttttttcttttttcttttttttggtcgggggggtgggagggttggattgaacagccagagggccccagagttccttgcatctaatttcacccccaccccaccctccagggttagggggagcaggaagcccagataatcagagggacagaaacaccagctgctccccctcatccccttcaccctcctgccccctctcccacttttcccttcctctttccccacagccccccagcccctcagccctcccagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctccccggggaatcctctctgccaatcctgcgagggtctaggcccctttaggaagcctccgctctctttttccccaacagacctgtcttcacccttgggctttgaaagccagacaaagcagctgcccctctccctgccaaagaggagtcatcccccaaaaagacagagggggagccccaagcccaagtctttcctcccagcagcgtttccccccaactccttaattttattctccgctagattttaacgtccagccttccctcagctgagtggggagggcatccctgcaaaagggaacagaagaggccaagtccccccaagccacggcccggggttcaaggctagagctgctggggaggggctgcctgttttactcacccaccagcttccgcctcccccatcctgggcgcccctcctccagcttagctgtcagctgtccatcacctctcccccactttctcatttgtgcttttttctctcgtaatagaaaagtggggagccgctggggagccaccccattcatccccgtatttccccctctcataacttctccccatcccaggaggagttctcaggcctggggtggggccccgggtgggtgcgggggcgattcaacctgtgtgctgcgaaggacgagacttcctcttgaacagtgtgctgttgtaaacatatttgaaaactattaccaataaagttttgtttaaaaaaaaaaaaaaaaaa

SEQ ID NO: 30 - protein, 351 aa, Homo sapiens
MGNAAAAKKGSEQESVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 31 - protein, 4 aa, Homo sapiens
  MISC_FEATURE (1)..(4): PKA-C-binding sequence in PKIB
RRNA

SEQ ID NO: 32 - protein, 649 aa, Homo sapiens
  MISC_FEATURE (1)..(649): Extracellular domain
HTNCPSDAPSSGTVDPQLYQEILKTIQAEDIKKSFRNLVQLYKNEDDTEISKKIKTQWTSLGLEDVQFVNYSVLLDLPGPSPSTVTLSSSGQCFHPNGQPCSEEARKDSSQDLLYSYAAYSAKGTLKAEVIDVSYGMADDLKRIRKIKNVTNQIALLKLGKLPLLYKLSSLEKAGFGGVLLYIDPCDLPKTVNPSHDTFMVSLNPGGDPSTPGYPSVDESFRQSRSNLTSLLVQPISASLVAKLISSPKARTKNEACSSLELPNNEIRVVSMQVQTVTKLKTVTNVVGFVMGLTSPDRYIIVGSHHHTAHSYNGQEWASSTAIITAFIRALMSKVKRGWRPDRTIVFCSWGGTAFGNIGSYEWGEDFKKVLQKNVVAYISLHSPIRGNSSLYPVASPSLQQLVVEKNNFNCTRRAQCPETNISSIQIQGDADYFINHLGVPIVQFAYEDIKTLEGPSFLSEARFSTRATKIEEMDRSFNLHETITKLSGEVILQIANEPVLPFNALDIALEVQNNLKGDQPNTHQLLAMASRLRESAELFQSDEMRPANDPKERAPIRIRMLNDILQDMEKSFLVKQAPPGFYRNILYHLDEKTSRFSILIEAWEHCKPLASNETLQEALSEVLNSINSAQVYFKAGLDVFKSVLDGKN

SEQ ID NO: 33 - protein, 20 aa, Artificial
  An artificially synthesized epitope peptode for preparing antibody
SARAGRRNALPDIQSSAATD

SEQ ID NO: 34 - protein, 20 aa, Artificial
  An artificially synthesized epitope peptode for preparing antibody
KEKDEKTTQDQLEKPQNEEK

SEQ ID NO: 35 - protein, 480 aa, Homo sapiens [sequence omitted]

SEQ ID NO: 36 - DNA, 3008 bp, Homo sapiens
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa